(19) United States
(12) Patent Application Publication
(10) Pub. No.: US 2025/0249270 A1
(43) Pub. Date: Aug. 7, 2025

(54) MULTI-OBJECTIVE CONTROLLER TO DELIVER PRECISION 3-D CONFORMAL THERMAL DOSE FOR MAGNETIC HYPERTHERMIA

(71) Applicant: The Penn State Research Foundation, University Park, PA (US)

(72) Inventors: Anil Chandra Attaluri, Middletown, PA (US); Nageshwar Arepally, Middletown, PA (US); Yash Lad, Middletown, PA (US); Shreeniket Pawar, Middletown, PA (US)

(21) Appl. No.: 19/046,654

(22) Filed: Feb. 6, 2025

Related U.S. Application Data
(60) Provisional application No. 63/550,199, filed on Feb. 6, 2024.

(51) Int. Cl. A61N 1/40

(52) U.S. Cl. CPC A61N 1/403

(57) ABSTRACT

A method and system are configured to facilitate heating of magnetically susceptible materials (MSM) positioned at or near a tumor site within a body. Embodiments can be configured to analyze tumor data, MSM distribution data, and heating rate data to simulate a temperature distribution in the tumor site to adjust application of a magnetic field to the MSM to maintain a first prescribed temperature at a tumor of the tumor site and a second prescribed temperature at healthy tissue adjacent the tumor site.

### CROSS-REFERENCE TO RELATED APPLICATIONS

This patent application is related to and claims the benefit of priority of U.S. Provisional Application 63/550,199, filed on Feb. 6, 2024, the entire contents of which is incorporated by reference.

### STATEMENT REGARDING FEDERALLY SPONSORED RESEARCH DEVELOPMENT

This invention was made with government support under Grant Nos. CA247290 and CA257557 awarded by the National Institutes of Health. The Government has certain rights in the invention.

### FIELD

Embodiments relate to methods and systems for cancer treatment using thermotherapy. For example, some embodiments of the methods and system for cancer treatment may utilize a controller configured to control the delivery of a precise thermal dose emitted from at least one heating device to facilitate magnetic hyperthermia via heating of magnetically susceptible materials, such as magnetic nanoparticles, injected into a patient's body. Some embodiments may be configured to utilize magnetic particle imaging (MPI) to control the thermal dose that is applied.

### BACKGROUND

Magnetic Nanoparticle Hyperthermia (MNPH) is a thermal therapy modality used to improve the effectiveness of chemotherapy and radiation therapy in the treatment of locally advanced solid tumors. MNPH involves the injection of magnetically susceptible materials (MSM) such as magnetic nanoparticles (MNP) into the tumor, followed by the application of an alternating magnetic field (AMF) to generate heat from MSM. The application of an AMF with a frequency range of 100-400 kHz causes hysteresis heating of MSM, which leads to an increase in temperature. This process triggers apoptosis or necrosis in tumor cells, thereby promoting their destruction.

### SUMMARY

We have determined that treatment planning and precise thermal delivery of MNPH in tissues face several significant challenges and are often employed in a way that results in damage to adjacent healthy tissue. For instance, there can be limited information on MSM distribution in the tissues, and the distribution of MSM within tissues may vary owing to factors such as the type of tumor, MSM/nanoparticle used, and the suspension solution. Additionally, there is limited spatial control of AMF. As MSM can be found in locations such as critical organs or injection sites close to healthy tissues, precise control over the spatial distribution of AMF is an important and significant challenge in MNPH.

To ensure patient safety while maximizing the heating efficiency of MSM, we have determined that appropriate safety limits for magnetic field strength and frequency can be set. According to Faraday's law, when an AMF passes through a conductive material, it generates eddy currents. In MNPH, human body tissues act as conductors, leading to the induction of these eddy currents in patients exposed to the AMF. These currents can result in non-specific heating within the body, potentially harming healthy tissues. Hence, establishing a patient-tolerable safety limit can help define the threshold below which the effects of eddy currents remain acceptable for patients.

We have found that, during MNPH, the temperature of the tumor should be maintained at a pre-selected temperature for a pre-selected period of time (e.g., above 43° C. for 20-60 min, etc.) while the temperature of adjacent healthy tissue should be maintained below a pre-selected temperature (e.g., at or below 39° C.) to help maintain the health of that healthy tissue.

The use of mathematical and computational modeling can be utilized to help facilitate the treatment planning and controls of MNPH to address these limitations. However, there remains unmet needs for accuracy and reliability of computational models used to predict the temperature distribution in tissue during MNPH, along with the development of control strategies to deliver precise therapeutic thermal dose in the tumor while minimizing the damage to adjacent healthy tissue.

We have determined that MPI can be used to determine the intratumor distribution of MSM with millimeter spatial resolution. While most MNPH models assume a homogeneous or mathematical distribution of MSM in tissues to ease computational cost, we have determined that integrating MPI images can improve the precision of computational models for better treatment planning and more reliable MNPH. We have further determined that a feedback control system that utilizes the MPI imaging can help facilitate the controller to control a specific thermal dose applied to the tumor while minimizing damage to adjacent healthy tissues. For example, the MPI images can be utilized in a feedback control loop used by the controller to update how a thermal dose is administered to a patient or animal so that the temperature of the tumor that the thermal dose is being directed can be maintained at a pre-selected temperature for a pre-selected period of time (e.g. above 43° C. for 20-60 min, above 45° C. for 20-50 min, etc.) while the temperature of adjacent healthy tissue should be maintained below a pre-selected temperature (e.g. at or below 39° C., at or below a temperature that is between 39° C., and 20° C., etc.) to help maintain the health of that healthy tissue.

In an exemplary embodiment, a method for cancer treatment comprises delivering magnetically susceptible materials to a tumor site; applying a magnetic field to the tumor site to generate heat from the magnetically susceptible materials, thereby heating the tumor site; and controlling the magnetic field using a controller operatively coupled to a magnetic field generation device that applies the magnetic field, wherein the controller is configured to maintain a first prescribed temperature at a tumor of the tumor site and a second prescribed temperature at healthy tissue adjacent to the tumor site.

In some embodiments, the first prescribed temperature is a temperature greater than or equal to 43° C. and the second prescribed temperature is a temperature that is less than or equal to 39° C.

In some embodiments, the step of applying the magnetic field to the tumor site to generate heat from the magnetically susceptible materials further comprises supplying a thermal dose to the tumor, wherein the thermal dose is defined by:

$$CEM 43 = {\int }_{t = 0}^{t = final} {B}^{( \frac{43 ° C . - T ( x , y , z , t )}{1 ° C .} )} dt$$

wherein CEM43 is the thermal dose, t is time, T(x, y, z, t) is a factor related to the temperature dependence of the rate of cell death, T is the temperature, and x, y, z are spatial coordinates.

In some embodiments the step of controlling the magnetic field using a controller further comprises providing the controller with a tumor boundary temperature set point; measuring a boundary temperature at a tumor boundary of the tumor site; comparing the tumor boundary temperature set point and the measured boundary temperature to obtain an error; and adjusting the alternating magnetic field based on the error.

In some embodiments, the tumor boundary temperature set point is 43° C. or 43.5° C.

In some embodiments, the boundary temperature is measured with a temperature probe positioned at the tumor boundary of the tumor site or a scanner device.

In some embodiments, the controller further comprises a fuzzy logic controller.

In some embodiments, the magnetic field is an alternating magnetic field that has a frequency of between 100-400 kHz.

In some embodiments, the controller is configured to utilize a data driven model to maintain the first prescribed temperature and the second prescribed temperature.

In some embodiments, the data driven model is trained using data collected during testing and clinical trials.

In an exemplary embodiment, a system for cancer treatment comprises a magnetic field generator configured to generate a magnetic field to heat magnetically susceptible materials located adjacent to and/or at a tumor site; a controller operatively coupled to the magnetic field generator so that the magnetic field is generated to maintain a first prescribed temperature at a tumor of the tumor site and a second prescribed temperature at healthy tissue adjacent the tumor site.

In some embodiments, the first prescribed temperature is a temperature greater than or equal to 43° C. and the second prescribed temperature is a temperature that is less than or equal to 39° C.

In some embodiments, the system further comprises a temperature probe operatively connected to the controller, wherein the temperature probe is configured to measure a boundary temperature at the tumor boundary and transmit the measured boundary temperature to the controller.

In some embodiments, the controller is configured to compare the measured tumor boundary temperature with a tumor boundary temperature set point to obtain an error and adjust how the magnetic field generator generates the magnetic field based on the error.

In some embodiments, the tumor boundary temperature set point is 43° C. or 43.5° C.

In some embodiments, the controller further comprises a fuzzy logic controller.

In some embodiments, the magnetic field is an alternating magnetic field that has a frequency between 100-400 kHz.

In an exemplary embodiment, a method for cancer treatment planning comprises delivering magnetically susceptible materials to a tumor site; imaging the tumor site with a first scanner to acquire tumor data, wherein the first scanner is a computed tomography scanner or a magnetic resonance imaging scanner; imaging the tumor site with a second scanner to acquire magnetically susceptible materials distribution data and heating rate data, wherein the second scanner is a magnetic particle imaging scanner; analyzing the tumor data, magnetically susceptible materials distribution data, and heating rate data to simulate a temperature distribution in the tumor site to adjust application of a magnetic field to the magnetically susceptible materials within a body having the tumor site to maintain a first prescribed temperature at the tumor site and a second prescribed temperature at healthy tissue adjacent the tumor site.

In some embodiments, a resolution of the magnetic particle imaging is 0.3-0.5 mm.

In some embodiments, the analyzing of the tumor data, magnetically susceptible materials distribution data, and heating rate data includes using finite element analysis, and the method further comprises segmenting the tumor data to obtain segmented tumor data; and segmenting the magnetically susceptible materials distribution data to obtain segmented distribution data.

In some embodiments, the method further comprises co-registering the segmented tumor data and the segmented distribution data.

In some embodiments, the first prescribed temperature is a temperature greater than or equal to 43° C. and the second prescribed temperature is a temperature less than or equal to 39° C.

In some embodiments, the method also comprises providing the controller with a tumor boundary temperature set point; simulating a boundary temperature at a tumor boundary of the tumor site; and comparing the tumor boundary temperature set point and the simulated boundary temperature to obtain an error for use in adjusting the application of the magnetic field to the magnetically susceptible materials within the body having the tumor site.

In some embodiments, the tumor boundary temperature set point is 43° C. or 43.5° C.

In some embodiments, the controller further comprises a fuzzy logic controller.

Other details, objects, and advantages of our process for screening, detecting, and treating infectious diseases, apparatuses for screening, detecting, and treating infectious diseases, systems for screening, detecting, and treating, and methods of making and using the same will become apparent as the following description of certain exemplary embodiments thereof proceeds.

### BRIEF DESCRIPTION OF THE DRAWINGS

The above and other objects, aspects, features, advantages, and possible applications of embodiments of the present innovation will be more apparent from the following more particular description thereof, presented in conjunction with the following drawings. Like reference numbers used in the drawings may identify like components.

FIG. 1 is a block diagram illustrating an exemplary embodiment of a system for thermotherapy treatment.

FIG. 2 is a block diagram illustrating an exemplary embodiment of a controller that can be utilized in an exemplary embodiment of the system for thermotherapy treatment.

FIG. 3 is a block diagram illustrating a PID controller cascaded with fuzzy logic to maximize the coverage index of the tumor.

FIG. 4 is a block diagram illustrating an exemplary system for thermotherapy treatment.

FIG. 5 is a block diagram illustrating another exemplary system for thermotherapy treatment.

FIG. 6 is a block diagram illustrating an exemplary embodiment of a computer device (CD) that can be utilized in embodiments of the system for thermotherapy treatment.

FIG. 7 shows an exemplary embodiment of a method for applying a thermotherapy treatment.

FIG. 8 shows an exemplary embodiment of a method for applying a thermotherapy treatment.

FIG. 9 shows mathematically calculated idealized MSM distributions—uniform (D1) and Gaussian (D2)—as well as the digitized image processing of an In-vivo MSM distribution (D3). The conservation of sphere volume and MSM domain in D3 guarantees accuracy.

FIG. 10 shows graphs illustrating the predicted temperature and CEM43 at a tumor boundary along with input power for a two-point controller.

FIG. 11 shows graphs illustrating the predicted temperature and CEM43 at a tumor boundary along with input power for a PID controller.

FIG. 12 shows graphs illustrating the predicted temperature and CEM43 at a tumor boundary along with input power without any constraint on the rate of change in amplitude.

FIG. 13 shows graphs illustrating the predicted temperature and CEM43 at a tumor boundary along with input power with a constraint on the rate of change in amplitude.

FIG. 14 shows graphs of PID controller input power and predicted temperature of a tumor for uniform (D1) distribution.

FIG. 15 shows graphs of PID controller input power and predicted temperature of a tumor for Gaussian (D2) distribution.

FIG. 16 shows graphs of PID controller input power and predicted temperature of a tumor for In-vivo (D3) distribution.

FIG. 17 is a graph showing results of CEM43 at a tumor boundary of a PID controller for three distributions (e.g., uniform, Gaussian, and In-vivo).

FIG. 18 is a block diagram illustrating an exemplary embodiment of a controller that can be utilized in an exemplary embodiment of the system for thermotherapy treatment.

FIG. 19 is a block diagram illustrating an exemplary embodiment of a controller that can be utilized in an exemplary embodiment of the system for thermotherapy treatment.

FIG. 20 shows mathematically calculated idealized MSM distributions in a human head tumor (entire portion of MSM distribution heated, middle and bottom portion of MSM distribution heated, and middle portion of MSM distribution heated).

FIGS. 21A-D shows graphs illustrating the predicted temperature and CEM43 at a tumor boundary along with input power for a PID controller.

FIGS. 22A-D shows graphs illustrating the predicted temperature and CEM43 at a tumor boundary along with input power for an MPC controller.

FIG. 23 shows geometry and the boundary conditions of an exemplary tumor embedded in healthy tissue. The tumor and MSM (e.g., MNP) are assumed to be ellipsoid with MNP at an off center from the center of the tumor. Healthy tissue is assumed to be a cuboid with tumor embedded. Boundary conditions included the top surface having convective cooling and the remaining surfaces having temperature boundary conditions.

FIG. 24 shows a domain probe to measure temperature and CEM43 at four points P1, P2, P3 and P4. Heat sources HS1, HS2, HS3 and HS4 correspond to points P1, P2, P3 and P4.

FIG. 25 shows a block flow diagram of exemplary architecture for fuzzy logic. The architecture may be expanded by dividing the regions into more than four regions.

FIG. 26A is a graph showing a volumetric heat source HS1 for a uniform distribution with maximum center temperature as 80° C.

FIG. 26B is a graph showing a volumetric heat source HS2 for a uniform distribution with maximum center temperature as 80° C.

FIG. 26C is a graph showing a volumetric heat source HS3 for a uniform distribution with maximum center temperature as 80° C.

FIG. 26D is a graph showing a volumetric heat source HS4 for a uniform distribution with maximum center temperature as 80° C.

FIG. 26E is a graph showing temperatures for P1, P2, P3, and P4, and maximum MNP, for a uniform distribution with maximum center temperature as 80° C.

FIG. 26F is a schematic illustration showing CEM43 contours after P1, P2 and P3 reaches the setpoint of CEM43=20 [min] and at the end of the treatment.

FIG. 26G is a graph showing CEM43 at the points P1, P2, P3 and P4 and at an offset of 2 mm from tumor boundary.

FIG. 27A is a graph showing the magnetic field strength of an MPC implementation for boundary temperature (Tb) of 43 [° C.].

FIG. 27B is a graph showing the temperature of an MPC implementation for boundary temperature (Tb) of 43 [° C.].

FIG. 27C is a graph showing the thermal dose of an MPC implementation for boundary temperature (Tb) of 43 [° C.].

FIG. 27D is a graph showing the magnetic field strength of an MPC implementation for boundary temperature (Tb) of 44 [° C.].

FIG. 27E is a graph showing the temperature of an MPC implementation for boundary temperature (Tb) of 44 [° C.].

FIG. 27F is a graph showing the thermal dose of an MPC implementation for boundary temperature (Tb) of 44 [° C.].

### DETAILED DESCRIPTION

The following description is of exemplary embodiments and methods of use that are presently contemplated for carrying out the present invention. This description is not to be taken in a limiting sense, but is made merely for the purpose of describing the general principles and features of various aspects of the present invention. The scope of the present invention is not limited by this description.

The various embodiments herein provide systems and methods for cancer treatment and treatment planning. It is contemplated that while cancer treatment may relate to the active application of thermotherapy, cancer treatment planning may relate to predictive analysis of applying thermotherapy.

In an exemplary embodiment for cancer treatment, the system delivers magnetically susceptible materials (MSM), such as magnetic nanoparticles (MNP), to a tumor site of a patient and uses an alternating magnetic field (AMF) to generate heat from the MSM, thereby heating the tumor site. Delivery of the MSM may be achieved in various ways, including without limitation, via localized (e.g., intravenous) delivery or systemic (e.g., oral) delivery. For example, the MSM may be injected via a local injection provided manually via at least one injector or by an automated injector, via an intravenous feed, via oral delivery via an item consumed by the patient or animal, or via another type of injection mechanism.

As used herein, the term “tumor site” may include a tumor or at least a portion of a tumor, adjacent healthy tissue, and a tumor boundary separating the tumor from the healthy tissue.

As used herein, the term “patient” may refer to any biological system to which treatment can be administered, including without limitation, humans and other animals (e.g., dogs, cats, horses, cows, cattle, etc.).

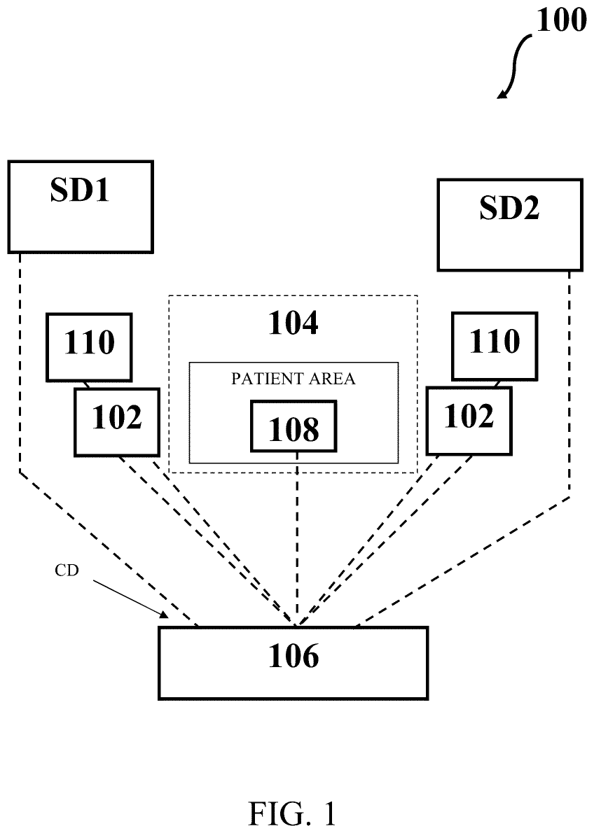

As seen in FIG. 1, the system 100 can include an AMF generator 102 configured to generate the AMF 104. The AMF generator 102 may be at least one magnet (e.g., electromagnet) configured to provide the AMF 104. The AMF 104 may be applied at a pre-selected frequency range. For example, the AMF 104 can be applied with a frequency range of 100-400 kHz or other suitable frequency range(s). The application of the AMF 104 within the pre-selected frequency range can be configured to cause hysteresis heating of the MSM injected into the patient, which can lead to an increase in temperature of the MSM and heating of tissue near the MSM via the heated MSM in proximity to that tissue. This heating process can trigger apoptosis or necrosis in cells of the tumor, thereby promoting their destruction.

The AMF delivery can affect the MSM so that the AMF and MSM work together to deliver an adequate thermal dose to the tumor site of the patient. The thermal dose is used to quantify the relationship between treatment efficacy and a target temperature as a function of time. In the most general form, pointwise thermal dose can be defined as:

$$D ( t ; x , y , z ) = {\int }_{0}^{t} f ( T ( τ ; x , y , z ) , τ ) dt . \qquad ( 1 )$$

The following is an expression of the total thermal dose achieved in a tumor:

$$CEM 43 = {\int }_{t = 0}^{t = final} {B}^{( \frac{43 ° C . - T ( x , y , z , t )}{1 ° C .} )} dt . \qquad ( 2 )$$

In the above equations, T represents temperature, t represents time, x,y,z represents spatial coordinates, and B is related to the temperature dependence of the rate of cell death.

CEM43 refers to the cumulative equivalent minutes wherein the pre-selected target heating temperature of the tumor is at least 43° C. for this particular embodiment (e.g., use of the 43° C. term in Equation (2) indicates that this is the pre-selected target heating temperature for the tumor). Similarly, CEM43 T90 refers to the cumulative equivalent minutes wherein the temperature of the tumor is at least 43° C. in 90% of the tumor. CEM43 T90 can be defined to represent the total duration of exposure to the pre-selected temperature of at least 43° C. that can contribute to a treatment's effectiveness. We have found that effective treatment for CEM43 T90 can be maintained for at least 60 minutes to heat the tumor to a desired temperature to provide a desired level of apoptosis or necrosis in cells of the tumor. Other pre-selected temperatures can be used that may adjust the duration of time that may be needed to provide a desired level of apoptosis or necrosis in cells of the tumor. Also, the size of the tumor, type of patient (e.g., animal type or sex of the patient) or type of tumor can result in a different pre-selected temperature or pre-selected duration range for application of that temperature being used to help provide the desired level of apoptosis or necrosis in cells of the tumor that is targeted for destruction or treatment.

For example, in some embodiments the AMF can be controlled to heat the tumor to a first prescribed temperature, such as a temperature greater than 43° C. However, the AMF being applied to such heating can also be controlled so that the heating heats the adjacent healthy tissue that is adjacent to the tumor to a second prescribed temperature, such as a temperature less than 39° C. Such first and second prescribed temperatures can be pre-selected to help ensure the destruction of the tumor while preserving the adjacent healthy tissue.

The system 100 can include a controller 106 operatively coupled to the AMF generator 102 and configured to control application of the AMF 104 in order to supply the thermal dose. In exemplary embodiments, the controller 106 can be configured as a proportional-integral-derivative (PID) controller that has multiple components. For instance, the PID controller of the controller 106 can include (1) a proportional controller configured to adjust the heating power in direct proportion to the difference between a simulated temperature and the target temperature for the tumor; (2) an integral controller configured to ensure that the control output aligns with a desired set-point temperature in a steady state; and (3) a derivative controller providing closed loop stability and enabling a faster ascent to the target while minimizing temperature overshooting that may affect the healthy tissue adjacent the target tumor.

In some embodiments, the PID controller output can be governed by the following equation:

$${U}_{ctrl} = {K}_{p} θ ( x , y , z , t ) + {K}_{i} {\int }_{0}^{t} θ ( x , y , z , t ) dt + {K}_{d} \frac{\partial θ ( x , y , z , t )}{\partial t} \qquad ( 3 )$$

In Equation (3), θ(x,y,z,t) represents the disparity between the temperature at the tumor boundary T(x,y,z,t) and the desired target temperature Tₛₑₜ. The parameters Kₚ(oe/K), Kᵢ (oe/(s·K)), and K_{d}(oe·s/K) denote the proportional, integral, and derivative gains of the PID controller, respectively. U_{ctrl} [oe] represents the controller output. The proportional response is influenced by the error, which is the difference between the tumor boundary temperature and the set point temperature, as well as the value of the proportional gain. Higher proportional gain values lead to a faster rise time but often result in higher overshoot and oscillations. If the proportional gain is excessively high, the response can become oscillating with increasing amplitude. The addition of the integral response can help mitigate the overshoot and oscillations.

In some embodiments, the MPC controller output can be governed by the following equations:

$${\dot{x}}_{k} = [ A ] {x}_{k} + [ B ] {u}_{k} \qquad ( 4 )$$

$${y}_{k} = [ C ] {x}_{k} + [ D ] {u}_{k} \qquad ( 5 )$$

$$[ A ] = [ \frac{∇ ( {k}_{i} ) ∇}{{ρ}_{i} {C}_{i}} - \frac{{ρ}_{b} {C}_{b}}{{ρ}_{i} {C}_{i}} {ω}_{b , i} ( t ) + \frac{{Q}_{met}}{{ρ}_{i} {C}_{i}} ] ; \qquad ( 6 )$$

$$[ B ] = [ \frac{{Q}_{MION}}{{ρ}_{i} {C}_{i}} ( t ) ] ; \qquad ( 7 )$$

$$[ C ] = [ 1 ; 1 ; 1 ; 1 ; 1 ; 1 ; 1 ] ; \qquad ( 8 )$$

$$[ D ] = [ 0 ; 0 ; 0 ; 0 ; 0 ; 0 ; 0 ] ; \qquad ( 9 )$$

For the MPC methodology, the input and output variables of the plant model are represented by Equations (4)-(9), where [A], [B], and [° C.] are the state, input, and output matrices of the plant, respectively; and [D] is the disturbance matrix. xₖ is the state vector, uₖ is the plant input variable vector, and yₖ is the plant output variable vector. ρ is the density [kg·m⁻³], Cp is the specific heat capacity [J(kg·K)⁻¹], k is the thermal conductivity [W(m·K)⁻¹], ρ_{b} is the blood density [kg·m⁻¹], C_{p,b} is the specific heat capacity of blood [J(kg·K)⁻¹], and ω_{b} is the microvascular blood perfusion [s⁻¹]. The subscript ‘i’ represents the domain under consideration (e.g. tumor, MSM and healthy tissue).

The system 100 can include at least one temperature probe 108 operatively coupled to the controller 106. In one embodiment, at least one temperature probe 108 is positioned at the tumor boundary and configured to measure the temperature at the tumor boundary and transmit the measured temperature values to the controller 106. In another embodiment, at least one temperature probe 108 is positioned at the tumor and configured to measure the temperature at the tumor itself and transmit the measured temperature values to the controller 106. In another embodiment, at least one temperature probe 108 is positioned at the adjacent healthy tissue and configured to measure the temperature at the adjacent healthy tissue and transmit the measured temperature values to the controller 106. The temperature probes of the present system may be fiber optic temperature probes.

The system 100 may further include at least one permanent magnet 110 configured to spatially confirm the heat within a tumor.

The system can also include one or more scanner devices SD1, SD2. Each scanner device can be communicatively connectable to the controller 106 and can be configured to provide at least one image of the patient for providing image data to the controller 106. The image data can be utilized by the controller to adjust how the AMF is generated and/or applied to the patient to provide a pre-selected thermal dose to the patient for destruction of a tumor. Each scanner device can be a computed tomography (CT) scanner, a magnetic resonance imaging (MRI) scanner, an MPI scanner, or other type of scanner device that can provide imaging of the patient to provide image data to the controller 106. In some embodiments, the first scanner device SD1 may provide imaging of the patient prior to any heating (e.g., before a magnetic field is applied), and the second scanner device SD2 may provide imaging of the patient prior to any heating and/or during heating (e.g., as a magnetic field is applied) such that the second scanner device SD2 may provide real-time imaging of the patient.

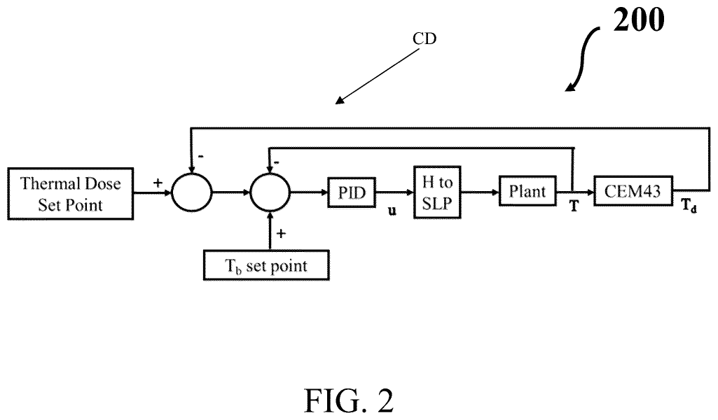

As seen in FIG. 2, the controller 200 of an exemplary embodiment of the system may include a thermal dose (TD) set point and a tumor boundary (Tb) temperature set point. The TD set point may correlate to the CEM43 thermal dose previously described (e.g., cumulative equivalent minutes wherein the temperature of the tumor is at least 43° C.). The Tb temperature set point may be 43° C. or other pre-selected temperature value to ensure the thermal dose is delivered while also maintaining appropriate temperatures at the adjacent healthy tissue. A calibration curve may be developed for correlation between H (AMF amplitude) and SLP (heating ability of the MSM/particles). The PID controller may send an updated signal to actuate the AMF system to adjust the H. The abovementioned calibration curve may be used to access the amount of correction and potential response. The system to be controlled is designated by the “plant” block.

In operation, the intensity of MSM heating may be adjusted based on the temperature measured at the tumor boundary by at least one temperature probe. In particular, after establishing the Tb temperature set point, the controller 200 may calculate error, which represents the difference between measured temperature and the Tb set point. The controller 200 may then alter application of the AMF based on the calculated error such that the controller 200 maintains a level of measured temperatures at the tumor boundary (and at the tumor itself) at a desired level during the course of treatment.

It is contemplated that to prevent overdosing, the controller 200 may be configured to deactivate the system after reaching the target thermal dose. It is further contemplated that the controller 200 may be configured to enforce an upper-temperature limit (e.g., a pre-selected upper threshold heating temperature maximum of 60° C., etc.) so as to avoid a large thermal gradient.

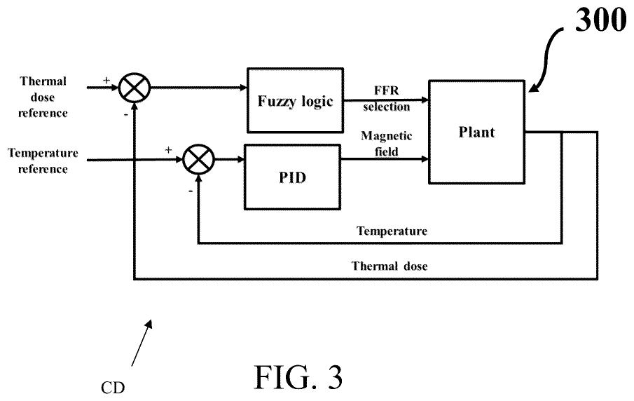

In some embodiments, as seen in FIG. 3, the controller 300 can be utilized as the controller 106 and be configured as a model predictive controller (MPC) and/or can include both a PID controller and a fuzzy controller.

The fuzzy logic controller component can be configured to mimic the way a knowledgeable human operator would apply a set of control rules appropriate to a given situation, which may overlap and even contradict each other. FIG. 3 illustrates a block diagram of a fuzzy logic control system which uses as its guide a set of pre-established response rules. The Quantisizer block takes the data from at least one temperature probe and converts it into a form of the fuzzy logic controller, while the PID controller calculates the control signal that is required to control the temperature of the tumor.

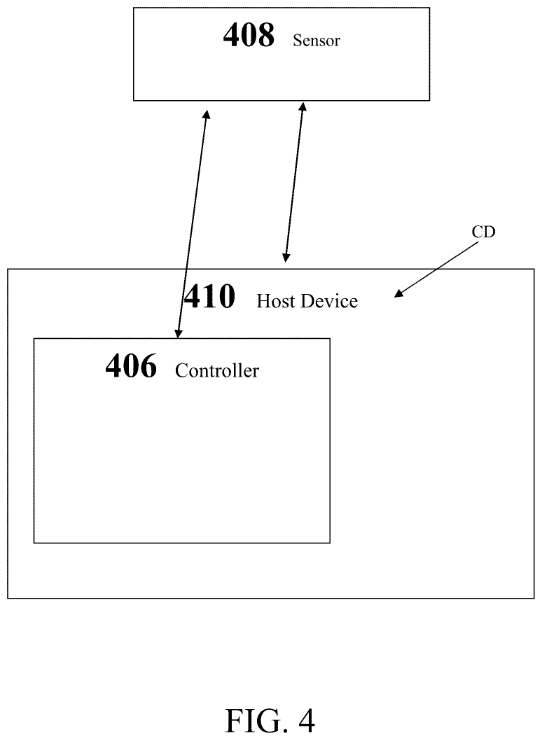

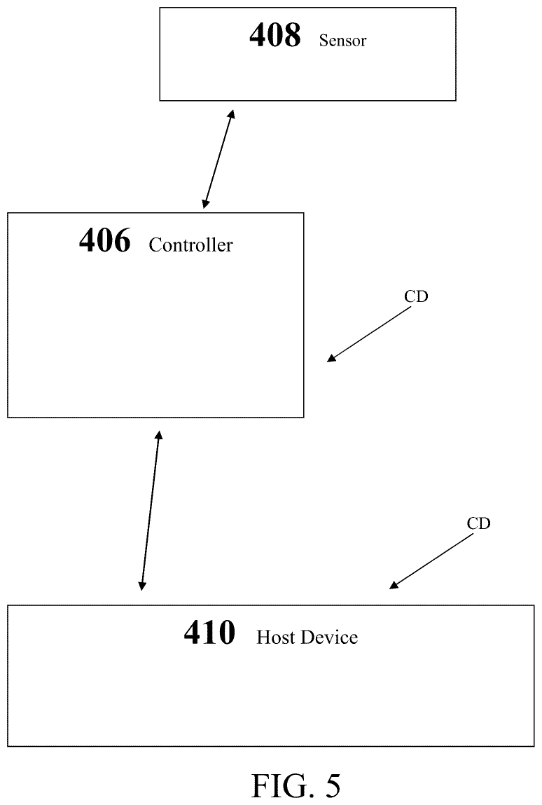

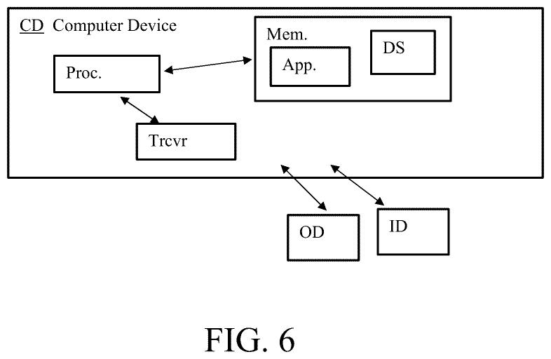

In some embodiments, as seen in FIG. 4, the temperature probes 408 may be hardwire connected to the controller 406, or the temperature probes 408 may be communicatively connected to the controller 406 via a network connection or wireless connection (e.g., internet connection, wide area network connection, near field communication connection, Bluetooth connection, etc.).

In some embodiments, a host device 410 can be configured to host the controller 406. The host device 410 may further be configured to receive data from the temperature probes 408 (sensor) for storage and analysis of the data. In some implementations, the host device 410 can be configured as a server or cloud-based service providing device for analysis and storage of the data obtained via the temperature probes 408, which can be sensors such as thermocouples (or other type of sensor) that can be communicated to a user via display device, which can be a tablet, smart phone, laptop computer, personal computer, or other type of terminal device. The display device can be effectuated via an application programming interface (API) and/or use of an application stored on the display device. It is contemplated that the host device 410 may comprise the display device, or the display device may be a separate device.

In other embodiments, the controller 406 can be configured to directly communicate with the one or more temperature probes 408 (e.g., thermocouples, MPI scanner, or other type of temperature sensor, etc.) and directly communicate with the AMF generator 102 to control application of the AMF 104. The controller 406 can also be communicatively connected to a host device 410 that can facilitate updating of software or control algorithm utilization by the controller 406. The host device 410 can also receive data from the controller 406 for use in obtaining more empirical data about use of the controller for updating control algorithms data for updating software to be run by the controller 406.

In some embodiments, the controller 406 may utilize a data driven model, such as an artificial intelligence model and/or a machine learning model, to aid in controlling and adjusting application of AMF in supplying a thermal dose to a tumor site. It is contemplated that the model may be trained using collected data, such as data collected during testing and clinical trials.

The controller 406 and/or host device 410 can be a computer device CD that can include a processor (Proc.) connected to a non-transitory memory (Mem.) and at least one transceiver (Trcvr) for forming communicative connections with one or more other devices. The at least one transceiver (Trcvr) can include a Bluetooth module and/or other type of transceiver unit (Trcvr) such that the temperature probes 408 and/or other input devices (ID) that may be configured for use with the controller 406 or host device 410. For instance, each temperature probe 408 can be communicatively connected to the controller 406 and/or host device 410 so that information collected by the temperature probes 408, such as temperature data, may be transmitted to the controller 406 or host device 410 so that data can be stored and analyzed, or displayed using the display device or other type of output device (OD) that can be communicatively connected to the computer device CD. Also, or alternatively, the temperature probes 408 can include a wireless local network transceiver (e.g., a Wi-Fi transceiver unit) or other type of wireless communication module so that information collected by the temperature probes 408, such as temperature data, may be communicated to the controller 406 or host device 410 so it can be stored and analyzed, or displayed using the display device.

The processor can be hardware (e.g., processor, integrated circuit, central processing unit, microprocessor, core processor, computer device, etc.), configured to perform operations by execution of instructions embodied in algorithms, data processing program logic, artificial intelligence programming, automated reasoning programming, etc. that can be defined by code stored in the memory. The processor can facilitate receipt, processing, and/or storage of readings from temperature probes 408 (e.g., MPI scanner, thermocouple, etc.) and/or control transmission of the collected data to the controller 406 or host device 410.

It should be noted that use of processors herein can include hardware, such as for example any one or combination of a Graphics Processing Unit (GPU), a Field Programmable Gate Array (FPGA), a Central Processing Unit (CPU), etc. The processor can include one or more processing or operating modules. A processing or operating module can be a software or firmware operating module configured to implement any of the functions disclosed herein. The processing or operating module can be embodied as software and stored in non-transitory memory, the memory being operatively associated with the processor. A processing module can be embodied as a web application, a desktop application, a console application, etc.

The memory (Mem.) can be a non-transitory computer readable memory configured to store data. Embodiments of the memory can include a processor module and other circuitry to allow for the transfer of data to and from the memory, which can include to and from other components of a communication system. This transfer can be via hardwired links or wireless transmission communication links. The communication system can include transceivers, which can be used in combination with switches, receivers, transmitters, routers, gateways, waveguides, etc. to facilitate communications between different devices via a communication approach or protocol for controlled and coordinated signal transmission and processing to any other component or combination of components of the communication system. The transmission can be via a communication link, which can be a wireless type of communication connection and/or a wired type of connection.

The computer or non-transitory machine-readable medium can be configured to store one or more instructions thereon. The instructions can be in the form of algorithms, program logic, etc. that cause the processor to execute any of the functions disclosed herein.

The processor can be in communication with other processors of other devices (e.g., a second external device, a computer system, a laptop computer, a desktop computer, etc.). An exemplary other device can be a Bluetooth enabled device, near field communication device, etc. Any of those other devices can include any of the exemplary processors disclosed herein as well as transceivers or other communication devices/circuitry to facilitate transmission and reception of wireless signals or other type of communicative connections.

Each computer device CD can also be configured to be connected to other input devices (ID) and output devices (OD). Examples of ID can include scanner devices (e.g., scanners), a microphone, a keyboard, a touch screen, buttons, sensors, detectors, or a pointer device. Examples of output devices can include a display, a printer, a speaker, or other type of output device.

Embodiments of the system and method for cancer treatment can be configured to provide real time monitoring of temperature at the tumor site.

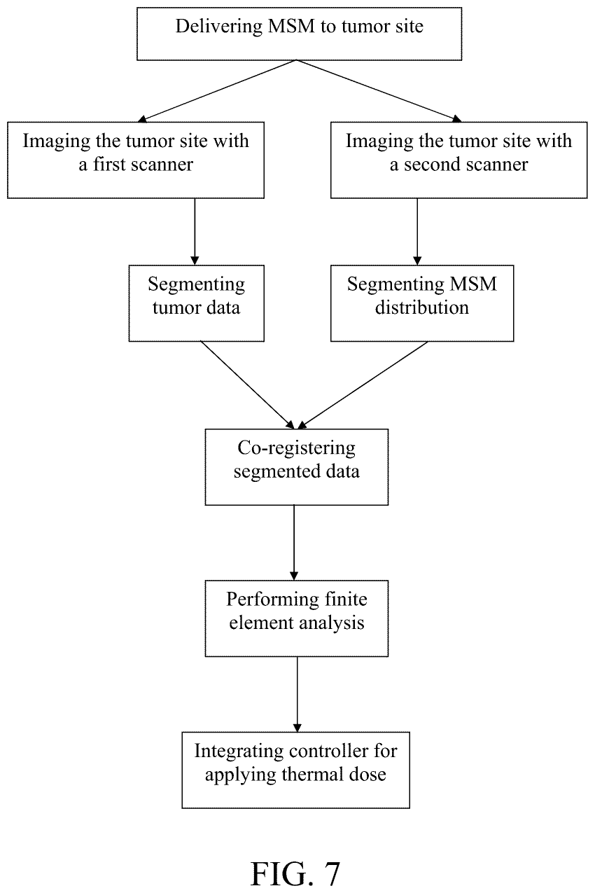

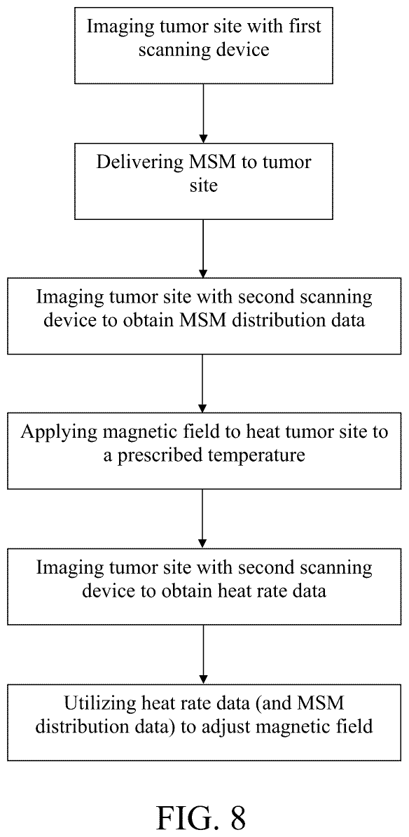

In an exemplary embodiment for cancer treatment planning (i.e., predictive analysis prior to actual application of thermotherapy), as seen in FIG. 7, after delivery of the MSM to a tumor site of a patient, the tumor site may be imaged using a first scanner SD1 to acquire tumor data. The first scanner SD1 may be a computed tomography (CT) scanner or a magnetic resonance imaging (MRI) scanner. The tumor data may be an image or a plurality of images of the tumor site captured by the first scanner.

The tumor site may also be imaged using a second scanner SD2 to acquire data representing MSM distribution at the tumor site. The second scanner may be a magnetic particle imaging (MPI) scanner. While previous cancer treatment planning may have assumed a homogeneous or mathematical distribution of MSM in tissues to ease computational cost, integrating MPI images can provide data representing MSM distribution at the tissue site which can improve the precision of computational models for better treatment planning.

Imaging data acquired via the first scanner SD1 and second scanner SD2 can be communicated to the controller 106 for utilization by the controller 106. Alternatively, such data can be provided to a host device 410 for use in updating the control algorithm of the controller 106 for application to heat to the patient-based o the image data acquired by the first and second scanners SD1 and SD2.

The MPI data may have a resolution between 0.3-0.5 mm. Such resolutions may predict temperatures within 1° C. of the highest possible MPI resolution (e.g., 0.15 mm). While high resolution MPI data can potentially improve the accuracy of the temperature predictions in treatment plans, it also increases image scanning time, data storage requirements, and computational time and resources to develop meaningful treatment plans. These demands potentially conflict with clinical realities for translation of MPI guided MNPH which favor fast or real-time treatment planning.

The first scanner data may be segmented such that a segmented tumor is digitally identified and separated from the remaining parts of the image(s). The second scanner data may similarly be segmented such that segmented MSM distribution is digitally identified and separated from the remaining parts of the image(s). Segmentation allows for the segmented MSM distribution and the segmented tumor to be co-registered. Co-registration is advantageous as it may be difficult to compare MPI and MRI/CT data separately and directly. For example, MPI provides intensity information about MSM, whereas MRI/CT provides information about the tumor itself. To address this challenge, markers may be used to align the coordinates of both the MPI and MRI/CT data. This procedure ensures that the spatial information of both modalities is accurately combined, allowing for the identification of MSM within the tumor and adjacent tissue.

The second scanner may further collect heating rate data. As the second scanner captures information about MSM, which act as a heat source, the second scanner may further capture information related to volumetric heat deposition. For example, a point of interest area may be extracted from the second scanner image representing MSM distribution, and when plotted, it shows as a point cloud. Utilizing a calibration curve, grayscale values may be transformed into heating rates.

It is contemplated that finite element analysis may be performed on the data captured by the first scanner (e.g., tumor data) and second scanner (e.g., MSM distribution and heating rate data). In particular, finite element analysis may be used to predict how the tumor and MSM distribution may react to real-world effects.

The finite element analysis may be modeled using Pennes bioheat equation:

$${ρ}_{i} {C}_{p , i} \frac{\partial T}{\partial t} - ∇ ( {k}_{i} ∇ T ) = {ρ}_{b} {C}_{p , b} {ω}_{b} ( {T}_{b} - T ) + {Q}_{met} \qquad ( 10 )$$

where C_{p,i} is specific heat capacity at constant pressure of tissue [J/kg K], Tis absolute temperature of tissue [K], kᵢ is thermal conductivity of tissue [W/m K], C_{p,b} is specific heat capacity at constant pressure of blood [J/kg K], ρᵢ is density of tissue [kg/m³], ρ_{b} is density of blood [kg/m³], ω_{b} is blood perfusion rate [1/s], T_{b} is temperature of blood [K], and Qₘₑₜ is metabolic heat source [W/m³].

A host device comprising suitable finite element analysis software may be used to facilitate simulations and analysis, enabling a comprehensive examination of the bioheat transfer process and its interaction with the tumor and MSM distribution.

A controller as described above with respect the system and method for thermotherapy treatment and as shown in FIGS. 3 and 4 may then be used with the finite element analysis to advantageously identify potential treatment scenarios and/or control application of the AMF via the AMF generator for providing the desired thermal does to the patient's tumor in a way that can help avoid damage to the patient's adjacent healthy tissue.

### EXAMPLES

### Example 1

This example relates to the use of hyperthermia temperatures using a PID controller. Through simulated experiments, we analyzed the performance of this thermal control method, taking into account factors such as nonlinear dynamics, system delay, uneven MSM/nanoparticle distribution, hardware and safety constraints, and computational demands.

Methods—Model Constraints: The main constraints on the model included: (i) Typically, it is necessary to restrict damage to healthy tissues. To address this requirement, temperature limits of 43° C. and 43.5° C. were set at the tumor boundary; (ii) MSM were injected intravenously, so the regions with higher concentrations were found. It was essential to enforce an upper-temperature limit of 60° C. at the injection site; and (iii) To prevent overdosing the target in the system with the controller, it was advisable to deactivate the system after reaching the targeted CEM43.

Methods—Two-point Controller: In the context of modulated-power heating, the intensity of MSM/nanoparticle heating was adjusted based on the temperature calculated at the boundary between the tumor and surrounding tissue. The heating power was adjusted between two levels: a higher power when the probe temperature, T_{b}, was less than 43.5° C., and a lower power when T_{b} exceeded 43.5° C. The heating algorithm was mathematically described in Equation (11):

$$H ( Tb ) = \{ \begin{matrix}150 oe , & if {T}_{b} > 43.5 ° C . \\ 50 oe , & if {T}_{b} \leq 43.5 ° C .\end{matrix} \qquad ( 11 )$$

The maximum power achievable by the HYPER system is restricted to 150 Oersteds (Oe). To expedite the increase in temperature within the tumor mass, the concentration of MSM was increased. Based on the observed temperatures at the tumor boundaries, the system was programmed to reduce the power to the lowest or highest necessary levels. Once CEM43 reached the predetermined set point, the system was shut off.

Methods—Thermal Damage Feedback PID Controller: We aimed to maintain a target temperature of 43° C. at the tumor boundary using a controller-based approach until the desired thermal dose was reached. To achieve this, we employed a proportional integral-derivative (PID) controller feedback loop that regulated both the boundary temperature (Tb) and thermal damage (TD), as illustrated in the block diagram in FIG. 2.

The input to the controller consisted of the recorded temperature at the tumor edge, set at 43° C., and the assessed thermal damage (TD) at that specific point. The controller then calculated the error, which represents the difference between the actual readings and the established targets. The aggregated error data were then fed into the controller. As a result, the output generated by the controller was incorporated into the model.

A model embodying a cascade PID control strategy was designed. This strategy capitalizes on feedback concerning both the thermal dose and the constrained tumor boundary temperature. The conversion process from H to SLP is anchored in a calibration curve, an innovative creation by Carlton, based on his experimental findings.

In practical scenarios, it may be unrealistic to expect an immediate alteration in equipment output. To ensure a reasonable and attainable adjustment pace, we introduced a constraint on the rate of change in the output per second, capping it at δH/δt=50 [Oe s] or 5 [mT s]. We manually tuned the PID controller.

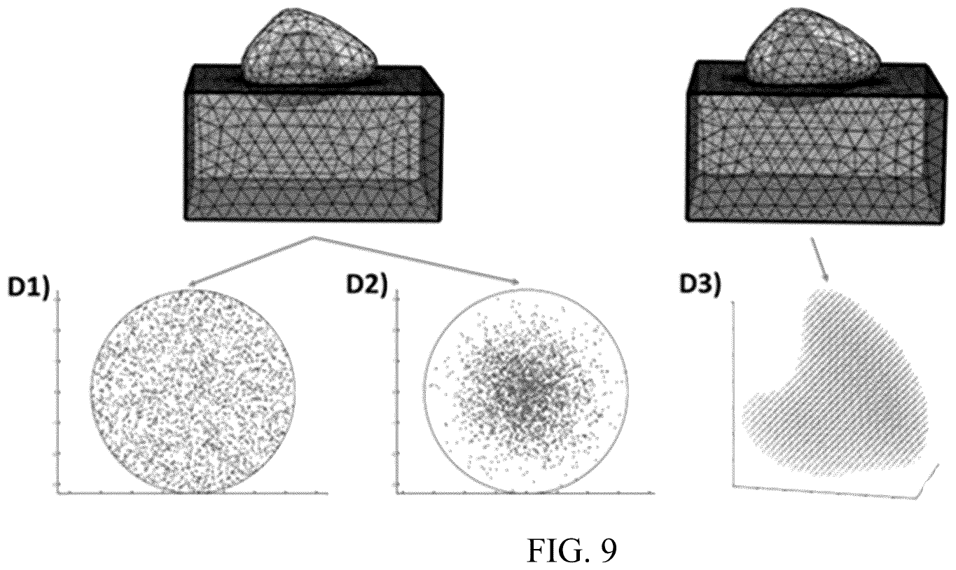

Methods—Mathematical and In-vivo MSM Distributions: To evaluate the robustness of the controller, we conducted PID tests by varying the MSM distributions. As shown in FIG. 9, the distributions used included uniform (D1), Gaussian (D2), and In-vivo (D3). D3 represents a realistic variation in the concentration profiles based on MPI scans of the mice. By analyzing the controller performance under these different distribution conditions, we were able to rigorously assess its stability and ability to handle changes that are representative of real-world scenarios.

Results—Modulating Power and PID Controller: In the evaluation of controller performance on a simulated nonlinear plant, the thermometric data from the simulated boundary between the tumor and healthy tissue were utilized to regulate the power during the heating process. This method involved a two-step power function, as outlined in Equation (5).

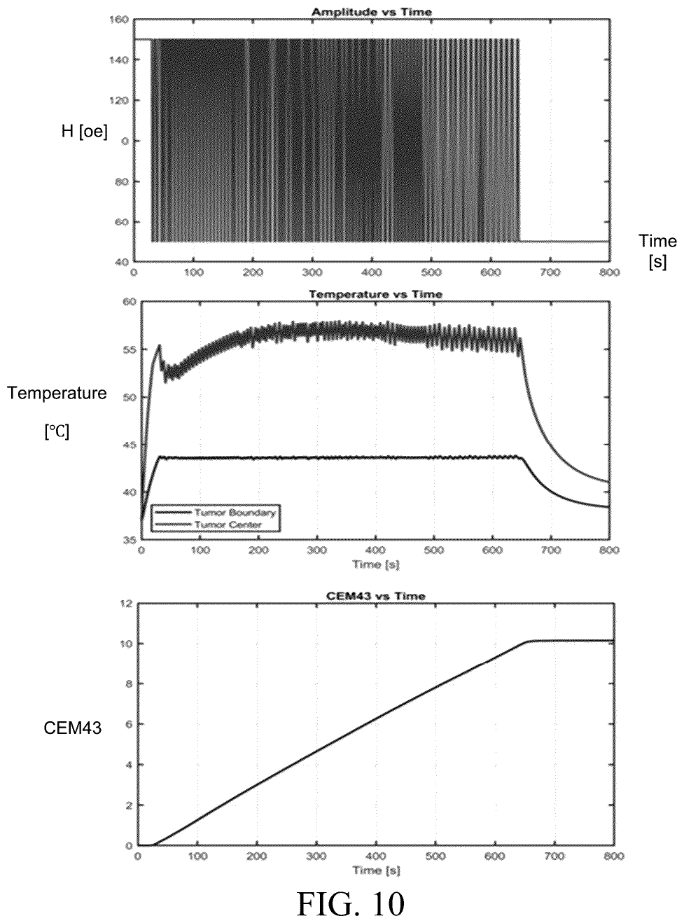

FIG. 10 illustrates the outcomes obtained using a two-point controller with a uniform distribution of the MSM, temperatures at the tumor center, and the tumor boundary. The targeted CEM43 was reached at the tumor boundary in just 648 [s], while the regulation of the input power AMF (H [oe]).

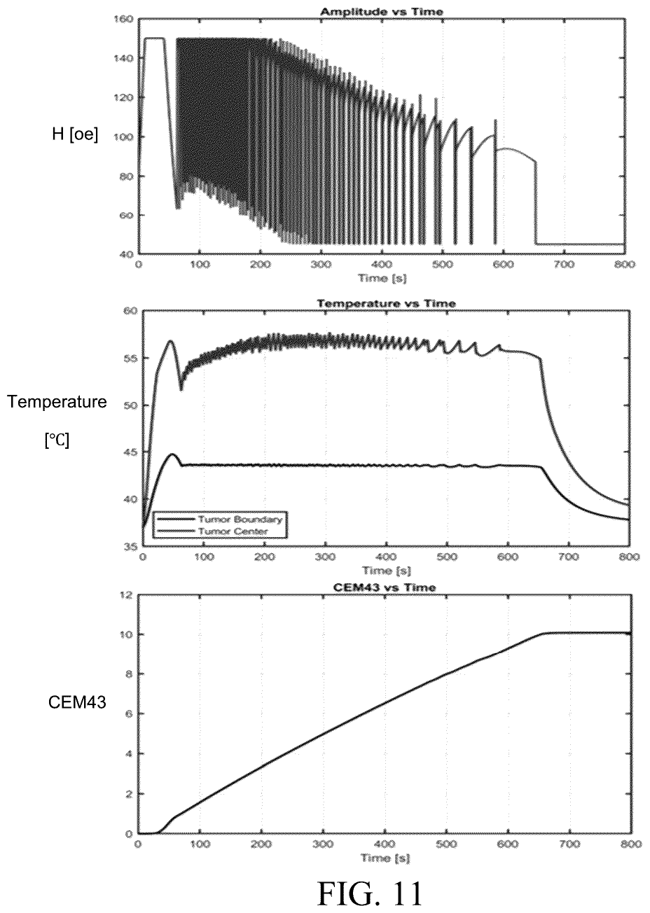

FIG. 11 shows the results obtained with a PID controller using a uniform distribution of the MSM prediction temperature. The system reached the desired CEM43 at the tumor boundary in 653 [s], only deviating from the two-point controller results by 5 seconds. PID started with maximum power and adjusted the peak amplitude as the targeted CEM43 was approached. For this simulation, the PID parameters were manually tunned to specific values: a proportional gain (Kp) of 5.0, an integral gain (Ki) of 0.5, and a derivative gain (Kd) of 0.01.

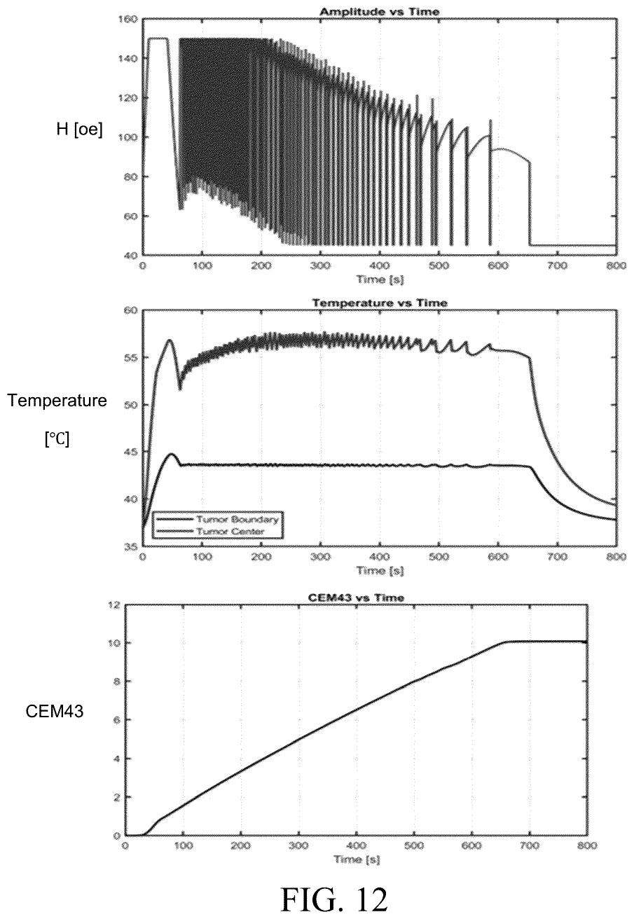

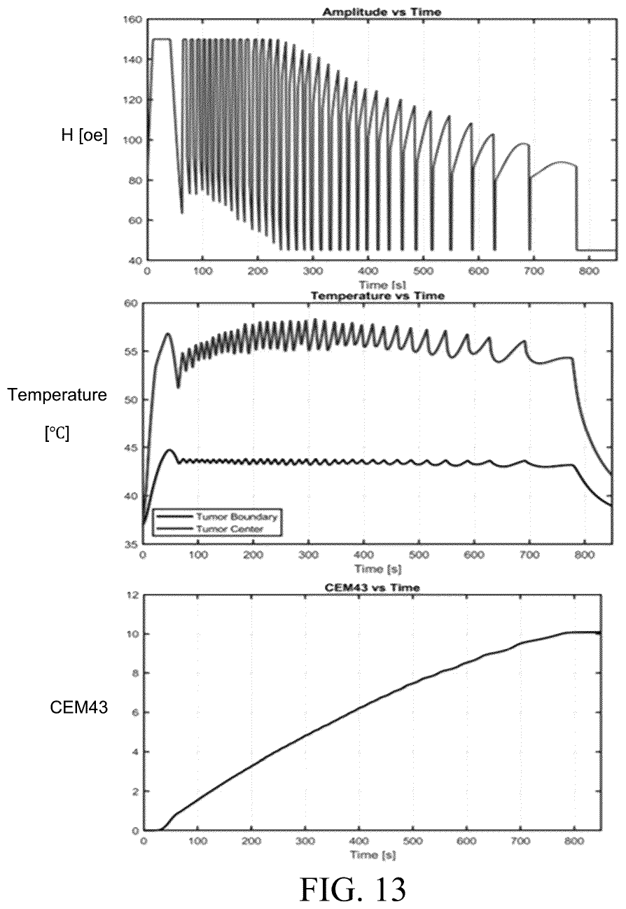

Results—Rate of Change in Amplitude Constraint: FIGS. 12-13 illustrate the results obtained by using a PID controller under different conditions: a) without limiting the amplitude's rate of change, reaching the targeted CEM43 in 653 [s], and b) with a restriction on the amplitude's rate of change (δH=50^{oe}ₛ), achieving the targeted CEM43 in 781 [s].

The examination in FIG. 12 indicates a rapid fluctuation in the controller power amplitude, which is implausible in practical systems because of the possibility of instability or physical constraints. This finding emphasizes the need to impose a constraint on the amplitude change rate, as shown in FIG. 13. Enforcing this limit leads to a more gradual change, making the system more feasible and safer, while slightly prolonging the treatment time needed to reach the targeted CEM43.

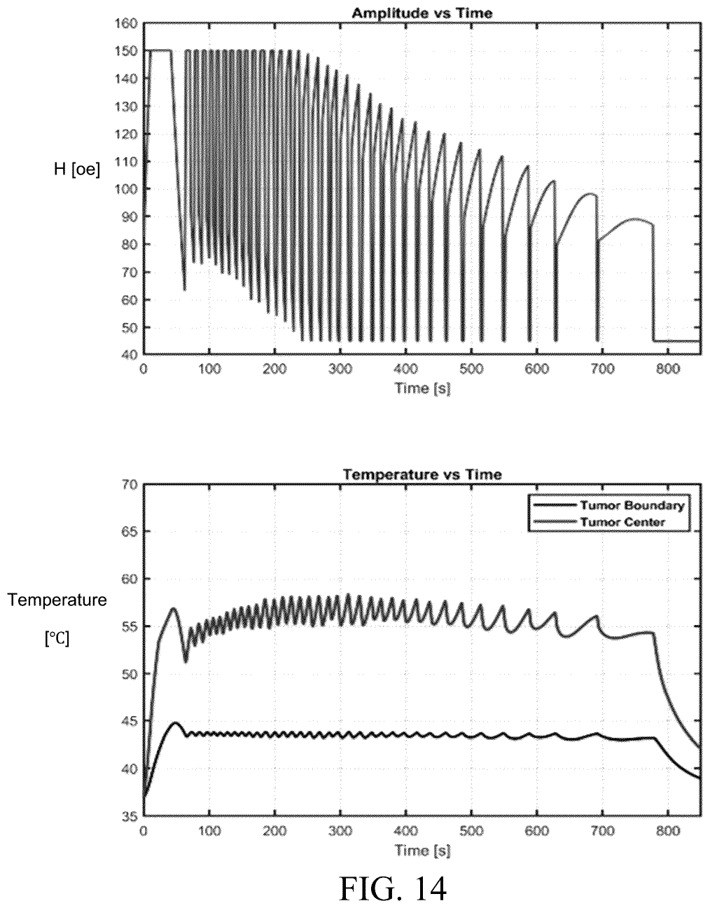

Results—PID Controller for Various Distribution: FIGS. 14-18 display several simulations of the PID controller's performance under various MSM distributions, highlighting the difficulties involved in regulating the temperature in tumors. In FIG. 14, the graph depicts the PID controller with a uniform distribution and the predicted temperature within the tumor, along with the applied input power H [oe], which leads to optimal performance and desirable outcomes.

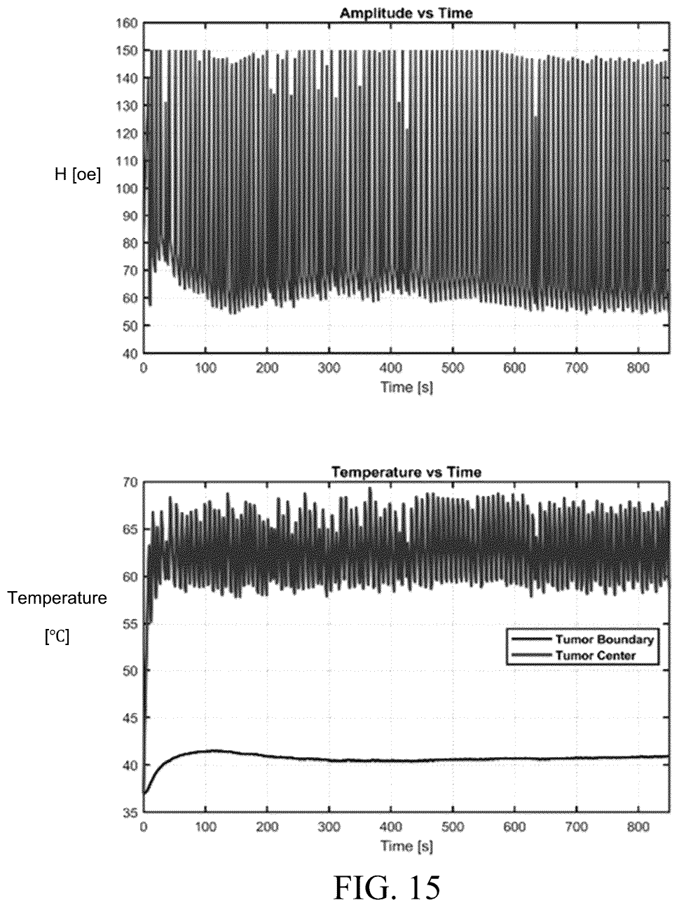

FIG. 15 presents a PID controller operating under a Gaussian distribution for the predicted temperature within the tumor, with applied input power H [oe] exhibiting instability. The constraint positioned at the center of the tumor prevents the boundary temperature from reaching the therapeutic threshold of 43° C., indicating a limitation of this control method in Gaussian distributions.

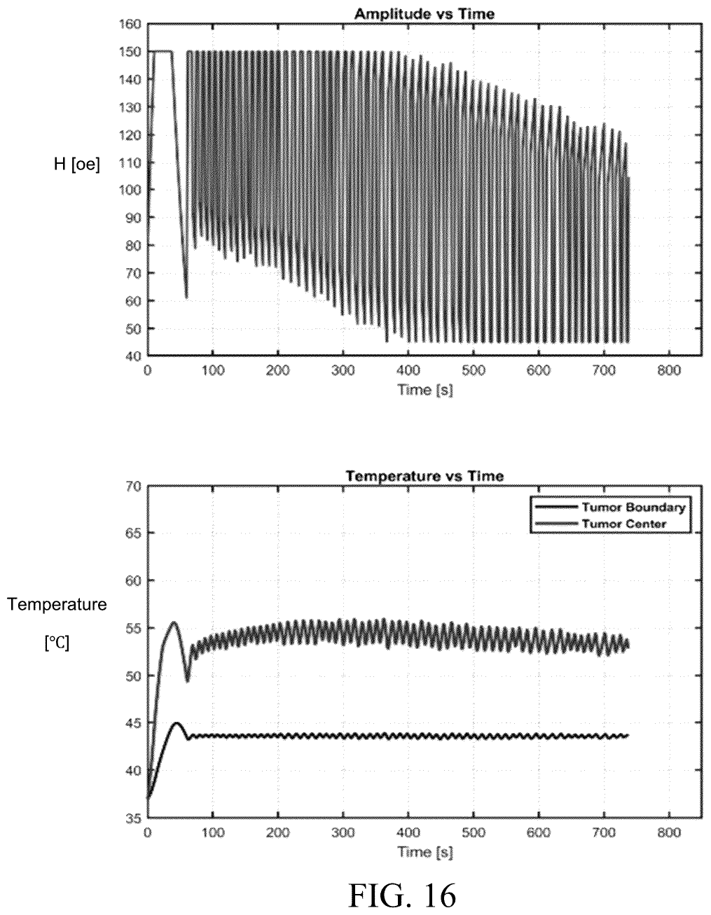

The implementation of the PID controller in the in vivo distribution scenario is shown in FIG. 16, where input power H [oe] is situated between D1 and D2 distributions, and the predicted tumor temperature. The simulation was limited to 750 [s] due to the significant computational memory required for in vivo simulations, which ultimately resulted in the system reaching its computational limit.

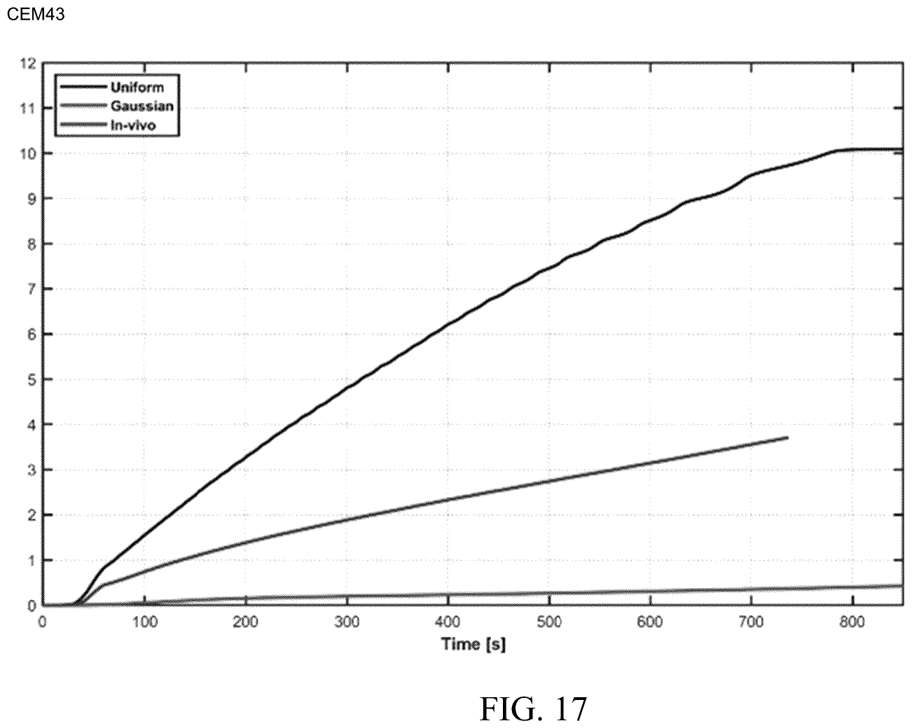

FIG. 17 depicts the percentage of volumetric CEM43 T90.

It is worth noting that the CEM43 T90 of D2 initially rose to a high value but subsequently experienced a gradual decline. In contrast, the difference between D1 and D3 was minimal, fluctuating around 6%. This observation pertains to the MSM distribution in the thermal response within the tumor volume.

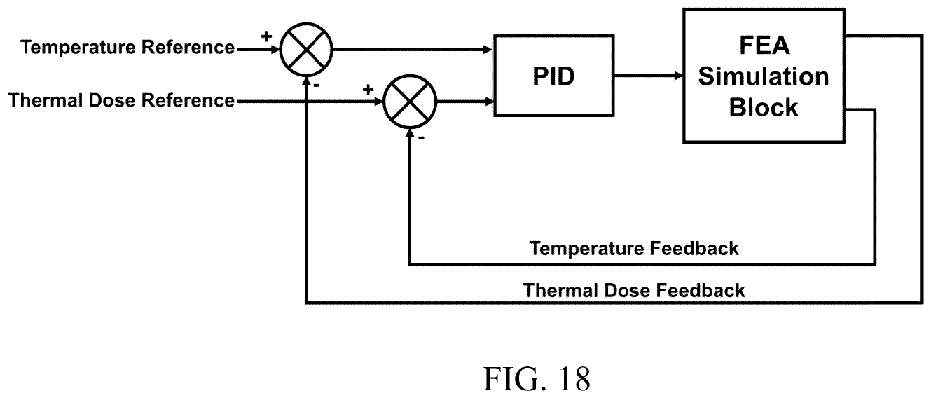

FIG. 18 shows the exploration of CEM43 at the tumor boundary beneath the muscle layer. Here, D2 registers a lower value due to the inability of the boundary temperature to reach the essential 43° C. mark for therapeutic effects. On the other hand, D1 successfully attained the targeted CEM43 within 781 seconds, demonstrating the efficiency of the applied control in that specific distribution. D3 presents an intriguing case where, despite having a high volumetric CEM43T90, the CEM43 measured at the tumor boundary below the muscle was significantly lower. This is because the majority of MSM s are concentrated above the muscle in the distribution observed in FIG. 9, resulting in fewer MSM within the tumor situated beneath the muscle. The PID controller parameters show in the Table 1.

TABLE 1
PID controller parameters for three distributions

| | Rise | Peak | Settling | Steady State | |
|---|---|---|---|---|---|
| | Time | Time | Time | Error | Overshoot |
| Distributions | [s] | [s] | [s] | [° C.] | (%) |
| Uniform | 29.5 | 48 | 69 | 0.5 | 2.64 |
| (D1) | | | | | |
| Gaussian | 42 | 112 | 300 | −2.6 | — |
| (D2) | | | | | |
| In-vivo | 28.5 | 44 | 67 | 0.5 | 3.22 |
| (D3) | | | | | |

| | Peak | Average | Average | CEM43 at Tumor |
|---|---|---|---|---|
| | Temp | T_{boundary} | T_{center} | Boundary |
| Distributions | [° C.] | [° C.] | [° C.] | (target = 10) |
| Uniform | 44.65 | 43.07 | 54.47 | 10 |
| (D1) | | | | |
| Gaussian | 41.48 | 40.64 | 62.54 | 0.426 |
| (D2) | | | | |
| In-vivo | 44.9 | 43.45 | 53.67 | 3.7 |
| (D3) | | | | |

Discussion: This example investigated the complex process of regulating the thermal dose within a simulated setting, highlighting the challenges arising from the nonlinear relationship between temperature and thermal dose, delayed responses of the system, and constraints imposed by actuators and healthy tissues. A challenge emerged from the spatial separation between the probe and heat sources generated by MSM, which led to a delay in the response of the system to control inputs. This delay necessitates an active control strategy that induces oscillatory power inputs, a phenomenon similar to that observed in ultrasound treatments.

FIGS. 12-13 clearly demonstrate the practical difficulties in implementing power-regulation systems. The unchecked power amplitude shown in FIG. 12, although theoretically effective, may conflict with the limitations of realistic systems and raises concerns regarding safety and hard-ware integrity. In contrast, the implementation of an amplitude change rate constraint, as depicted in FIG. 13, although it prolongs the duration required to reach the targeted CEM43, may represent a more sustainable and safe approach.

FIGS. 14-17 illustrate the performance of the PID controller in relation to the varying MSM distributions. The instability encountered in the Gaussian distribution (FIG. 15) and the computational limitations in the in-vivo scenario (FIG. 16) highlight the complex interaction between the MSM distribution and effective thermal control. Despite these challenges, our models suggest a promising trend: PID-controlled power modulation has the potential to enhance the tumor heating efficiency across diverse MSM distributions while minimizing damage to healthy tissues, as shown in FIGS. 14-16.

The critical inquiries into the spatial distribution of MSM and their implications for treatment success elicited by the uneven CEM43 values within the tumor, particularly beneath the muscle layer, as observed in FIG. 17, suggest the necessity for more evenly dispersed MSM distributions. The sparse presence of MSM in these lower regions may have contributed to suboptimal thermal dose outcomes. However, practical implementation presents a challenge because of the unsuitability of heterogeneous MSM deposition. Previous studies have highlighted the unsuitability of heterogeneous MSM deposition due to leakage and distribution along the needle track. This realization, coupled with the adaptability of the HYPER system, proposes a superior strategy compared to scanned or focused heating modalities. It offers more effective thermal dose delivery, even amidst less-than-ideal MSM distributions, thereby minimizing potential damage to healthy tissues.

### Example 2

This example relates to the use of hyperthermia temperatures using PID and MPC controllers. Through simulated experiments, we analyzed the performance of this thermal control method, taking into account factors such as nonlinear dynamics, system delay, uneven MSM/nanoparticle distribution, hardware and safety constraints, and computational demands.

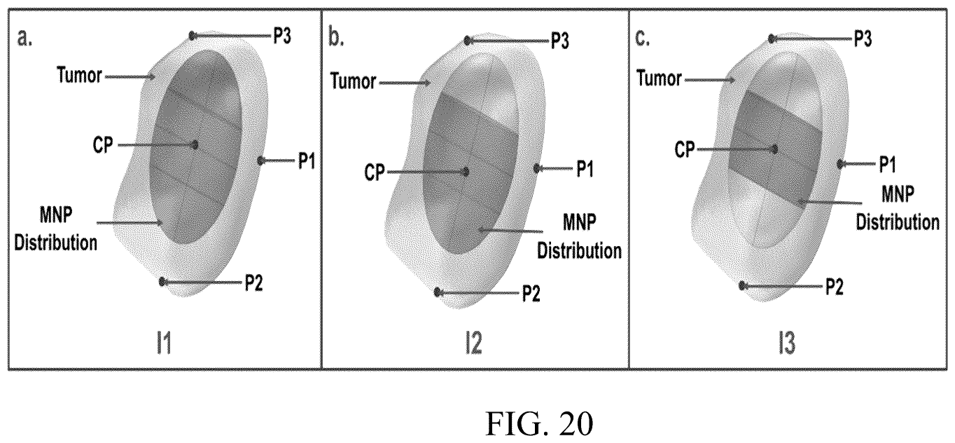

FIG. 20 shows the MSM distribution within the tumor modeled as an ellipsoid using FEA software. a. Iteration 1 (I1): The entire distribution heated to cover the maximum thermal dose delivery. b. Iteration 2 (I2): The bottom portion of the distribution was heated to cover the untreated tumor margins near probe 2. c. Iteration 3 (I3): The central portion of the distribution was heated to cover the untreated tumor margins near probe 1.

Methods—Model Constraints: The main constraints on the model included: (i) Typically, it is necessary to restrict damage to healthy tissues. To address this requirement a temperature limit of 43° C. was set at the three tumor boundary probes (P1, P2 and P3); (ii) MSM within the tumor were modeled as an ellipsoid. Within this ellipsoid, the MSM were modeled using a uniform distribution. It was essential to enforce an upper-temperature limit of 60° C. at the injection site; and to prevent overdosing the target in the system with the controller, it was advisable to deactivate the system after reaching the targeted CEM43.

Methods—Thermal Damage Feedback PID Controller: We aimed to maintain a target temperature of 43° C. at the tumor boundary using a controller-based approach until the desired thermal dose was reached. To achieve this, we employed a proportional integral-derivative (PID) controller feedback loop that regulated both the boundary temperature and thermal damage as illustrated in the block diagram in FIG. 18.

The input to the controller consisted of the recorded temperature at the three tumor boundary probes (P1, P2 and P3), set at 43° C., and the assessed thermal damage (TD) at these points. We manually tuned the PID controller.

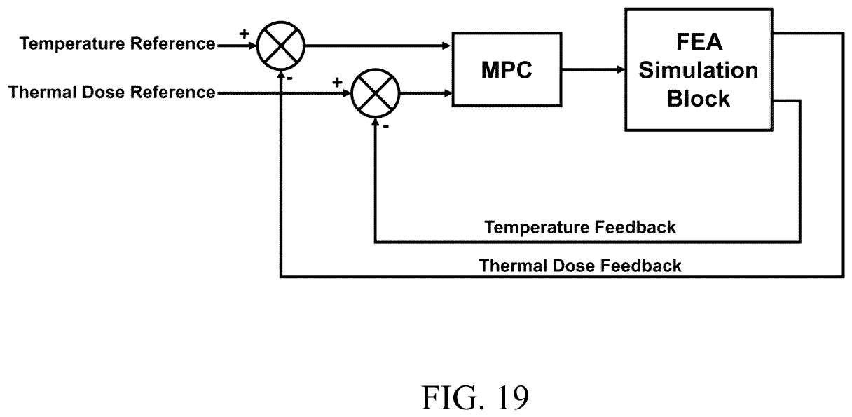

Methods—Thermal Damage Feedback MPC Controller: We aimed to maintain a target temperature of 43° C. at the tumor boundary using a controller-based approach until the desired thermal dose was reached. To achieve this, we employed a model predictive controller (MPC) feedback loop that regulated both the boundary temperature and thermal damage, as illustrated in the block diagram in FIG. 19.

The volumetric heat source applied to MSM s is considered as the control action and the temperature and CEM43 measured at tumor boundary are considered as the output variables. Weights were introduced on the constraints to assign higher importance to the probes that were more likely to reach the setpoint depending on their position. The prediction horizon, N, was selected as 100 and the control horizon, Nu, as 3, based on the intricacy of the problem being solved. This selection provided better controller performance by achieving reduced overshoot and aggressiveness simultaneously.

Results—PID Controller: In the evaluation of controller performance on a simulated nonlinear plant, the thermometric data from the simulated boundary between the tumor and healthy tissue were utilized to regulate the power during the heating process.

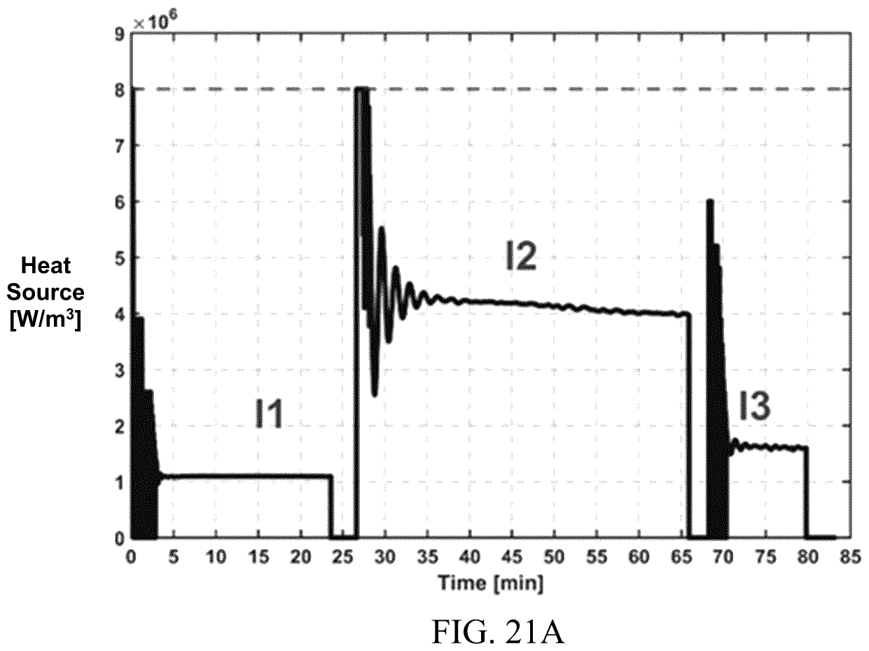

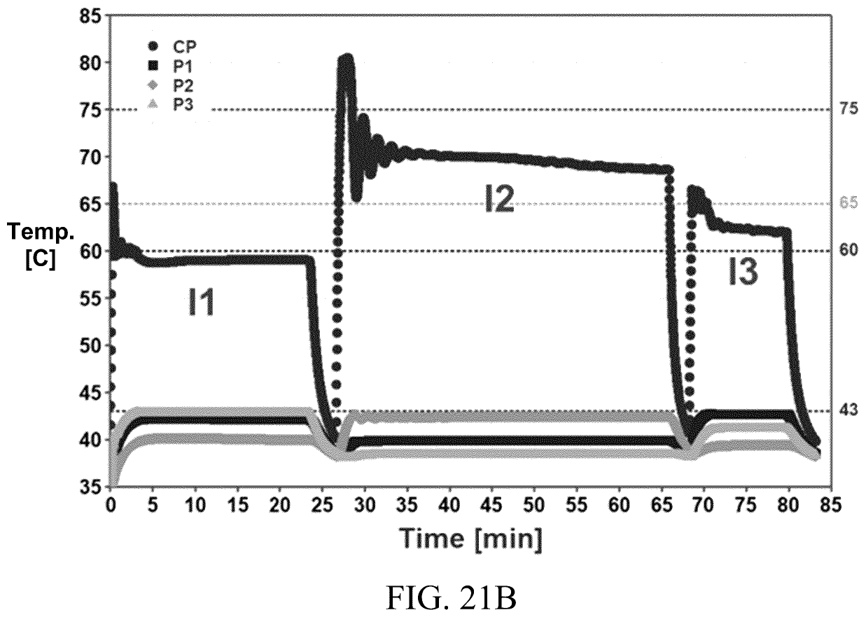

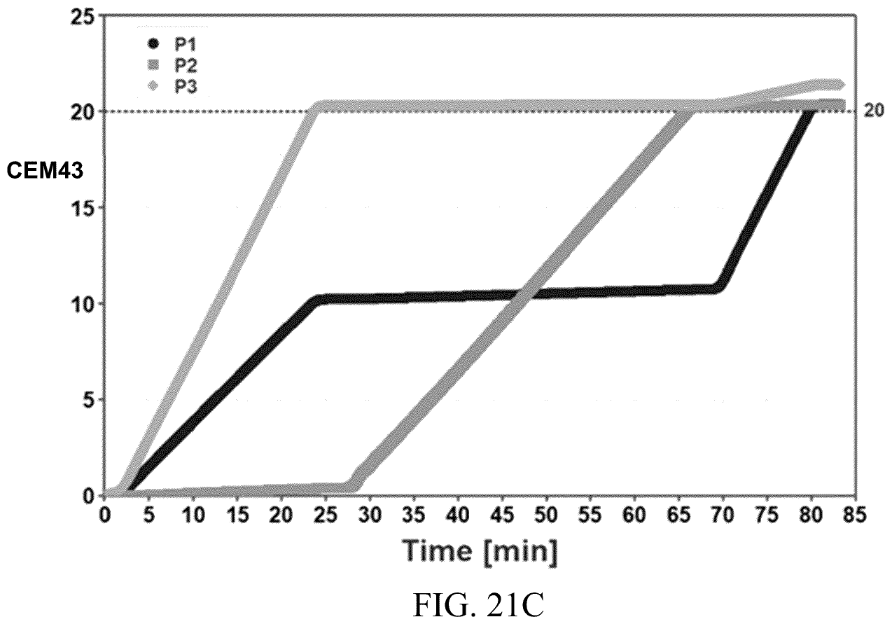

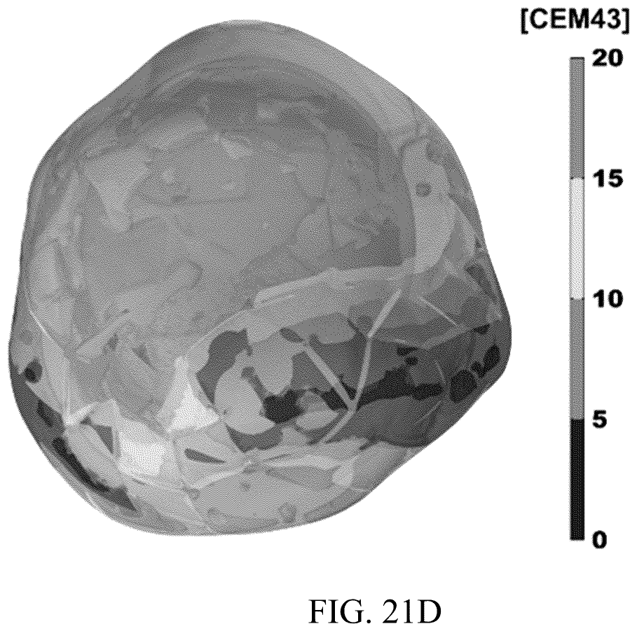

FIGS. 21A-D illustrate the outcomes obtained using thermal damage feedback based PID controller with a uniform ellipsoid distribution of the MSM, temperatures at the tumor center (CP), and the tumor boundary (P1, P2 and P3). A considerable number of initial controller output oscillations occurred at the beginning of each iteration with PID control. These initial oscillations in the controller output increased the treatment time and indicated a poorly tuned controller. The PID gains were further tuned manually: proportional gain K_{P}=0.7 [W (m³·° C.)⁻¹], integral gain K_{I}=5×10⁻⁷ [W/(m³·s·° C.)]; derivative gain K_{D}=9×10⁻⁸ [W·s/(m³·° C.)]. A target CEM43 of 20 [min] was achieved for all the boundary probes in 80 [min].

Results—MPC Controller: In the evaluation of controller performance on a simulated nonlinear plant, the thermometric data from the simulated boundary between the tumor and healthy tissue were utilized to regulate the power during the heating process.

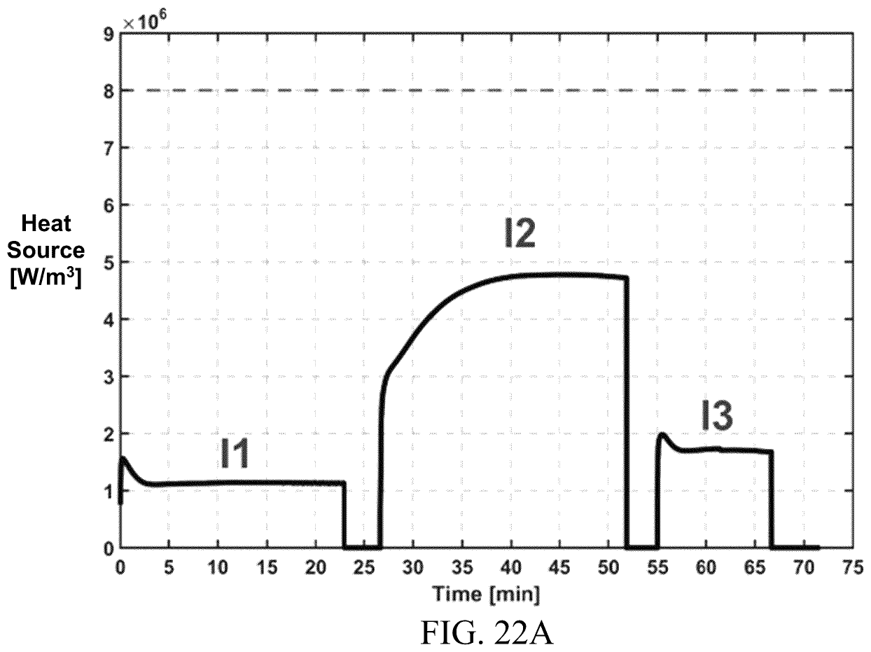

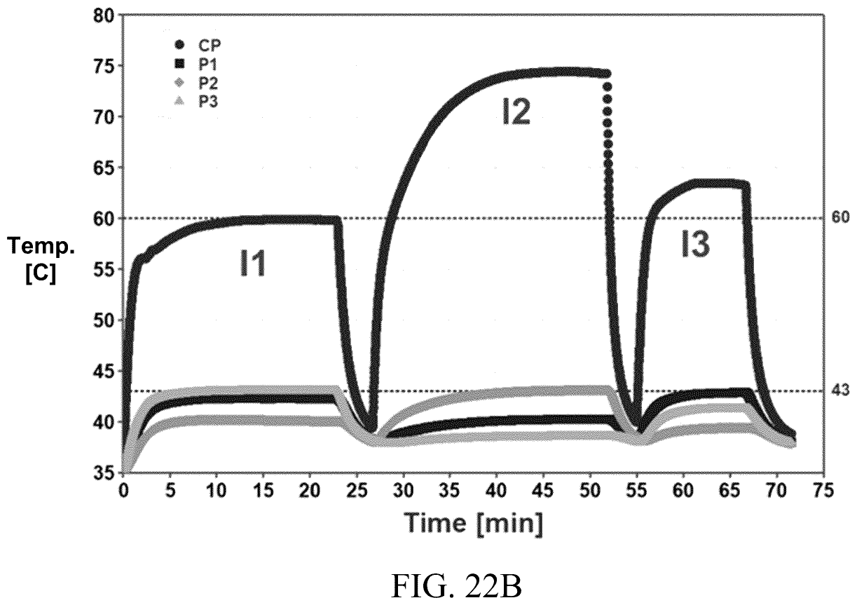

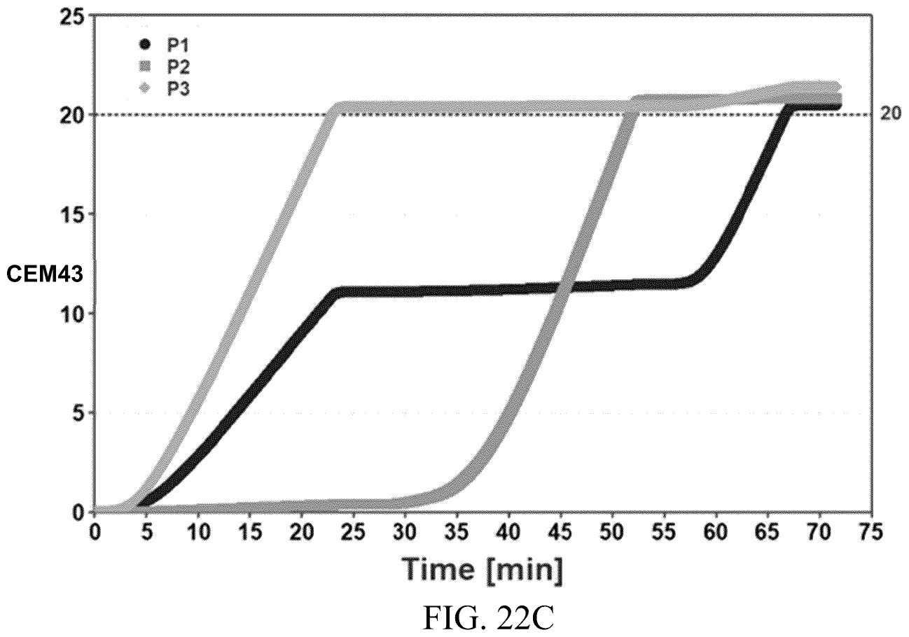

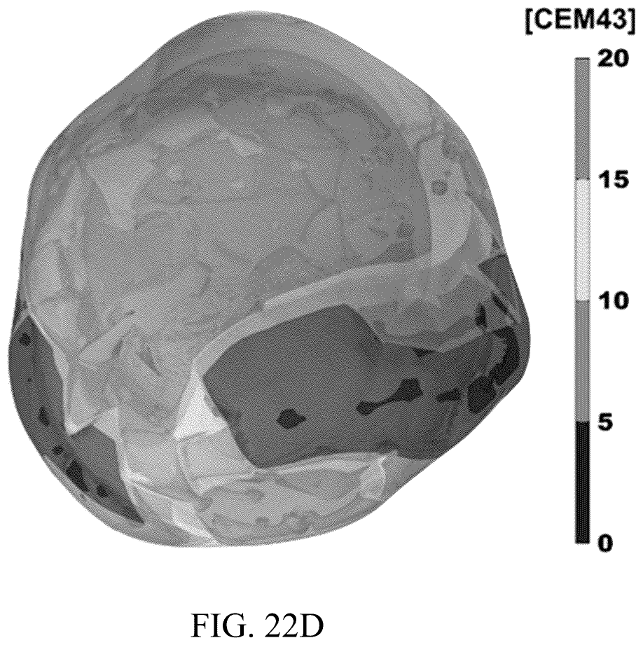

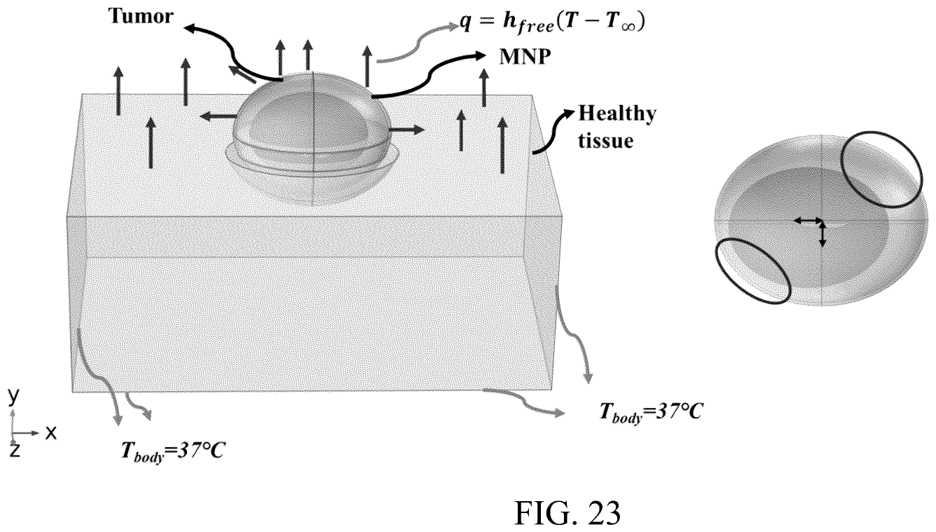

FIGS. 22A-D illustrate the outcomes obtained using thermal damage feedback based MPC controller with a uniform ellipsoid distribution of the MSM, temperatures at the tumor center (CP), and the tumor boundary (P1, P2 and P3)., the MPC controller eliminated high amplitude oscillations, and the controller output maintained a non-fluctuating response for most of the treatment time, with minimal need for manual tuning. In addition, the MPC reached the thermal dose for I2 in 30 [min] whereas the PID controller required 40 [min]. P1, P2, and P3 reached the target CEM43 of 20 [min] achieved in 67 [min]. Overall, the MPC achieved a better response, without requiring manual adjustments, than did the PID controller.

### Example 3

A cascaded PID controller with a fuzzy logic controller is developed to maximize coverage index based on temperature and thermal dose feedback. The PID controller is constrained to maintain the temperature at the tumor boundary at 46 [° C.]. Fuzzy logic is employed to select the region for heating with dynamic adjustment in FFR depending on the tumor boundary temperature and thermal dose. The objective of the controller is to optimize the tumor coverage index while minimizing damage to healthy tissues.

It is noted that Example 3 had similar hardware and safety constraints as Example 1.

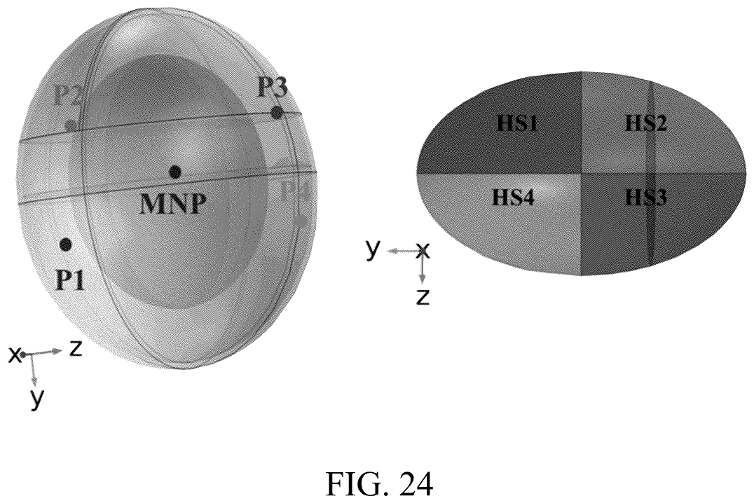

To maximize the tumor coverage index, the heat source was partitioned into four segments: HS1, HS2, HS3, and HS4, as illustrated in FIG. 24. To assess the feasibility of increasing CI four regions were selected and then a set of rules were implemented. Four-point probes P1, P2, P3, and P4 corresponding to HS 1, HS2, HS3, and HS4, respectively as depicted in FIG. 3, were utilized to monitor temperature and CEM43. To evaluate the potential damage to healthy tissue, a surface probe was positioned to assess CEM43.

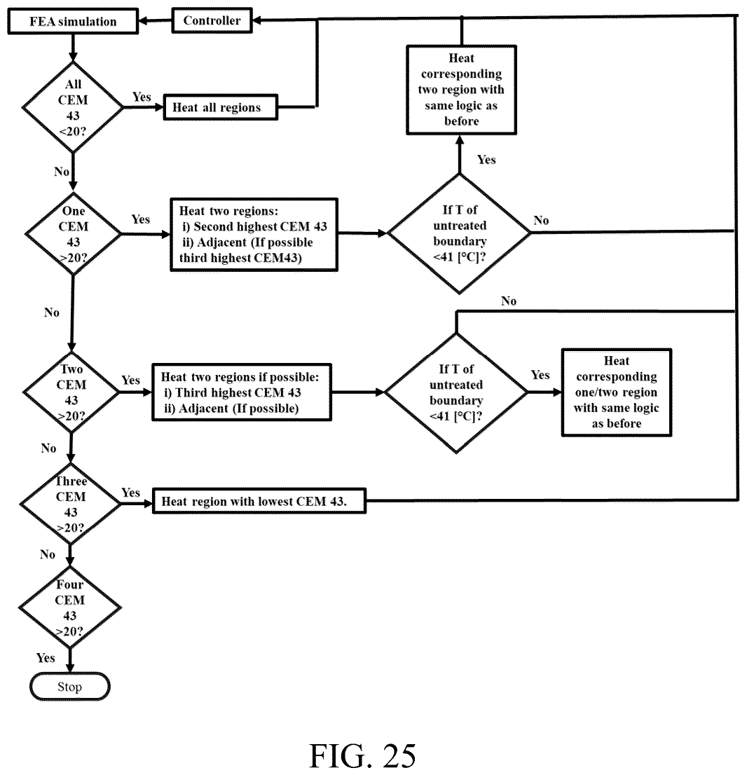

The following set of rules was employed for the fuzzy logic implementation as shown in FIG. 25. Initially, all regions were heated until the CEM43 of the one-point probe reached the predetermined setpoint. Subsequently, when the CEM43 of one probe attained the setpoint, two zones were selected: one corresponding to the CEM43 nearest below the setpoint, and the second corresponding to the CEM43 second nearest to the setpoint, if feasible; otherwise, the region corresponding to the lowest CEM43 was chosen. After the CEM43 of two probes reached the setpoint, the third region was selected such that the zone was below the setpoint. It was then verified whether the probe with the lowest CEM43 was adjacent to the selected zone, and if affirmative, the corresponding zone was activated. Once three probes reached the setpoint, the final zone corresponding to the probe that had not attained the setpoint was activated. Additionally, during the treatment, if the temperature of the boundary probe that had not reached the CEM43 setpoint decreased below 41 [° C.], the corresponding zone was activated to heat, employing the same fuzzy logic as shown in FIG. 3. This can be extended into smaller regions.

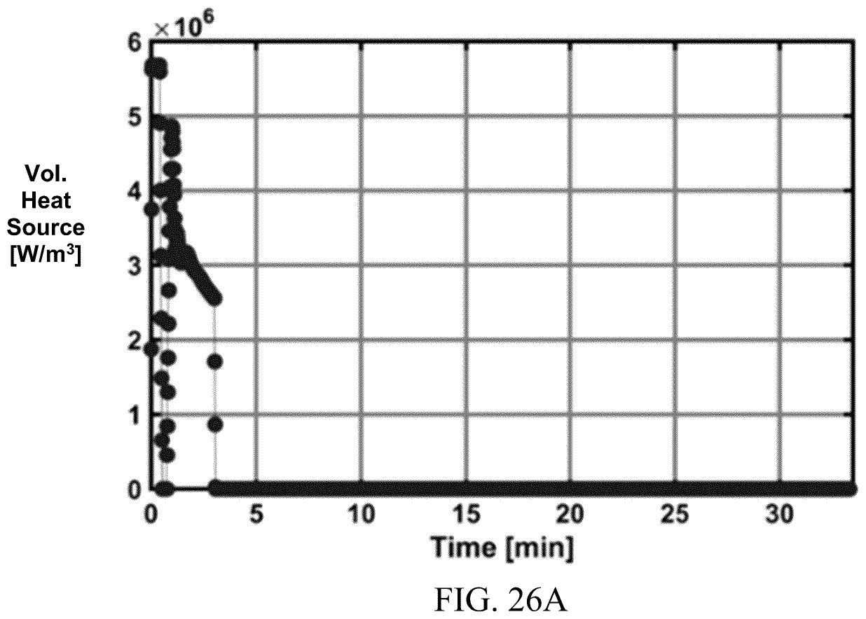

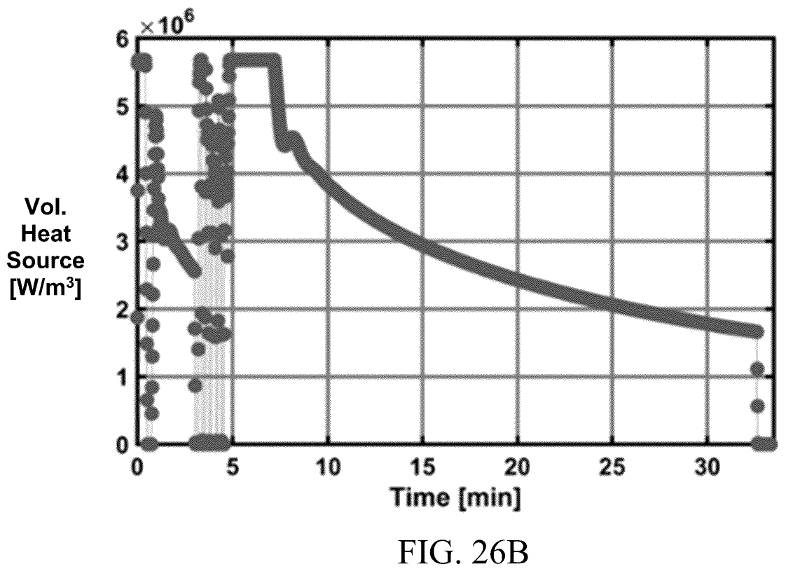

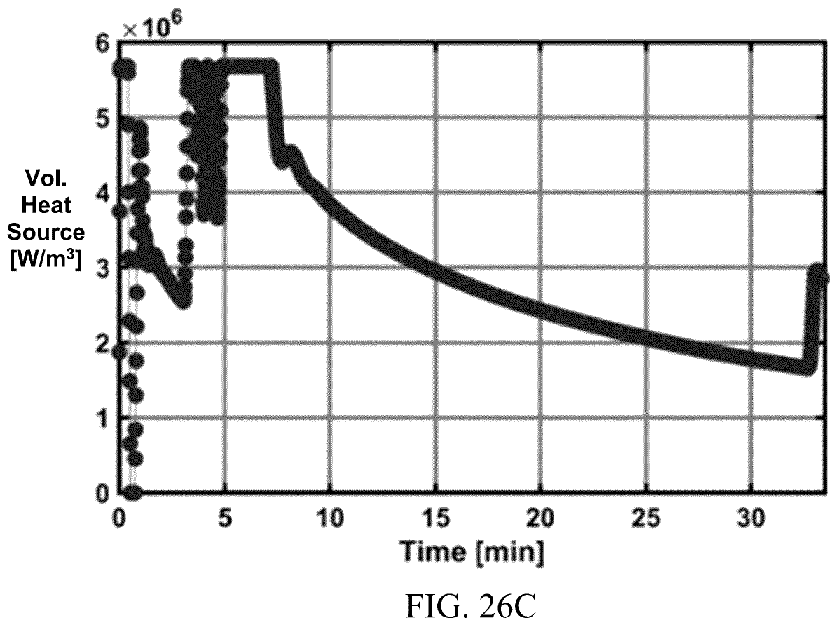

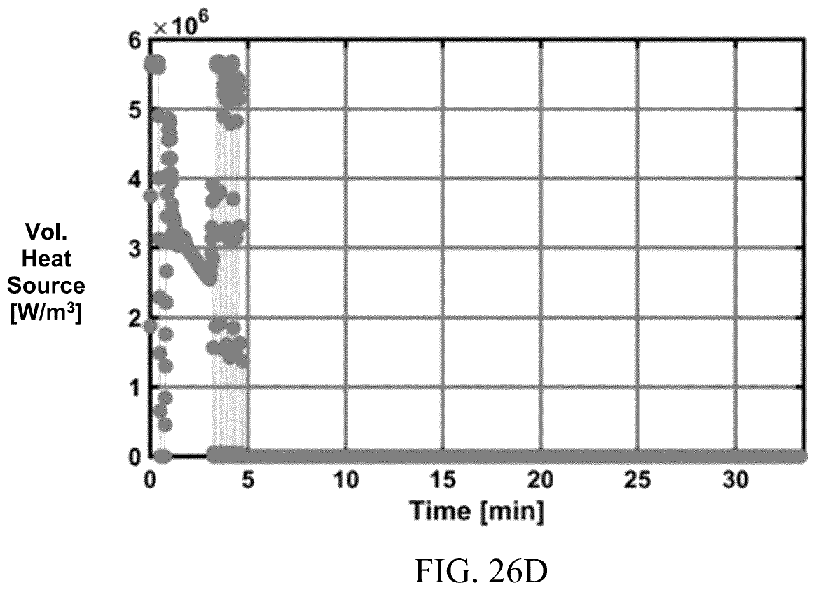

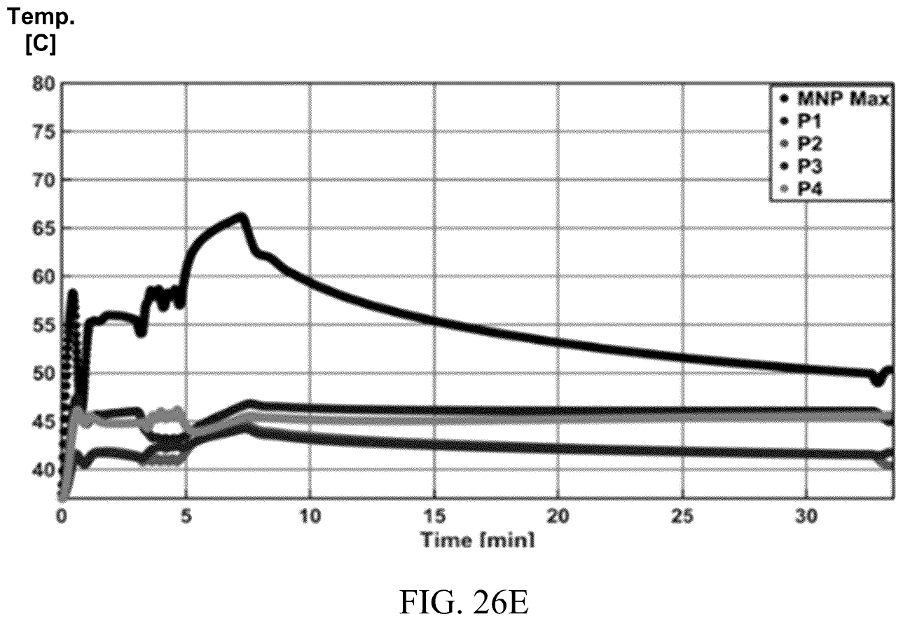

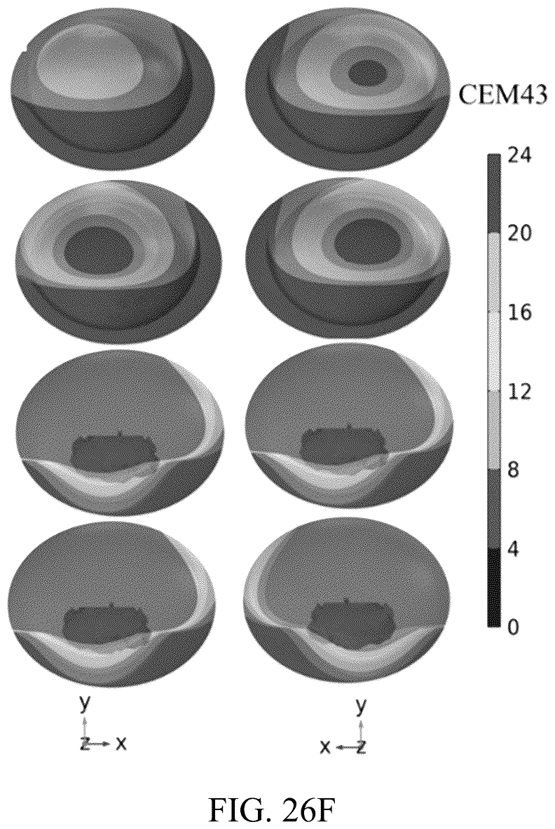

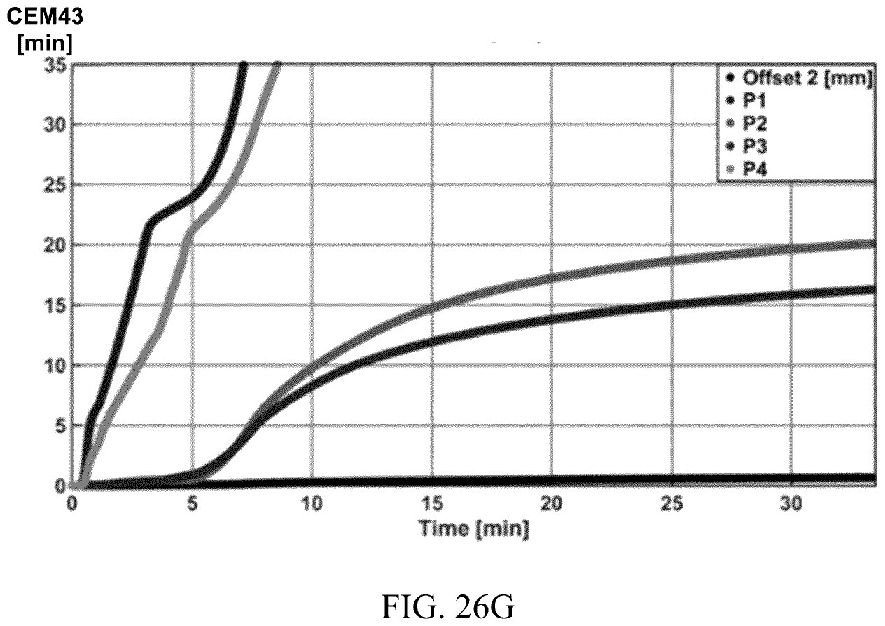

The temperature at the tumor boundary was maintained at the setpoint of 46 [° C.] at P1 as shown in FIG. 26E. The CI after P1 had reached the CEM43 setpoint of 20 [mins] was approximately 46 [%] at 181 [s],. Next, P4 reached the CEM43 setpoint with a CI of approximately 55 [%] at 285 [s]. By the end of the treatment, the coverage index (Tumor with CEM43>20 [min]) was approximately 91 [%] as shown in FIG. 26F.

TABLE 2
Tumor coverage index increases after reheating the tumor.

| | Uniform | Gaussian |
|---|---|---|
| | coverage index | coverage index |
| P1 CEM43 = 20 [min] | 45.77 | 33.30 |
| P2 CEM43 = 20 [min] | 54.85 | 40.49 |
| P3 CEM43 = 20 [min] | 91.04 | — |
| At end of treatment 33.33 [min] | 91.28 | 75.29 |

### Example 4

It is noted that Example 4 had similar hardware and safety constraints as Example 1.

The goal of the controller was to reach the target thermal dose with minimum treatment time and non-specific heating.

Constraints were that the temperature of tumor boundary (T_{b}) must be between 43-45 [° C.], the tumor maximum temperature (Tₘₐₓ) must be less than 80 [° C.], and the off-target (muscle, fat, and skin) temperature (T_{off}) must be below 39 [° C.]. The homogenization of variables with respect to an initial temperature of 37 [° C.] yielded ΔT_{b}, ΔTₘₐₓ, and ΔT_{off} of 6-8, 43 and 2 [° C.].

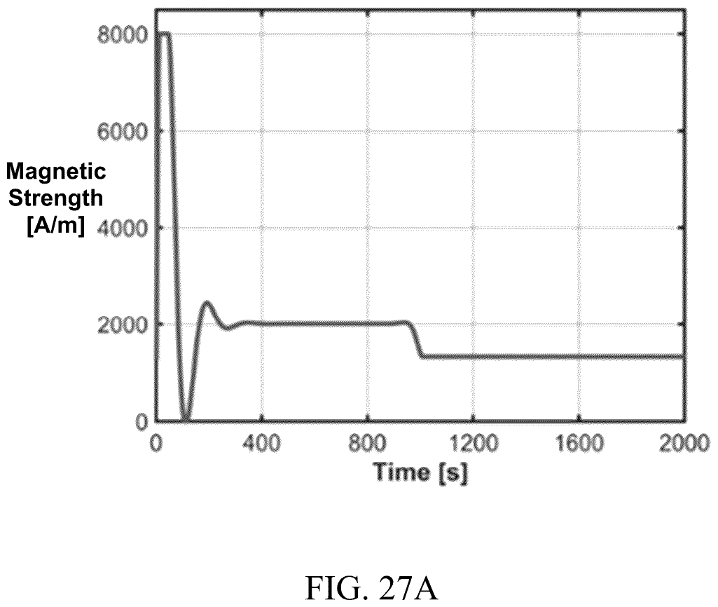

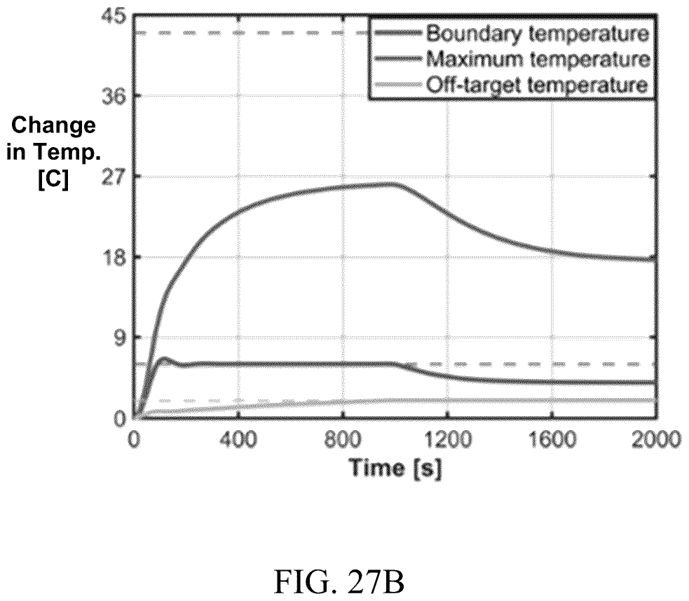

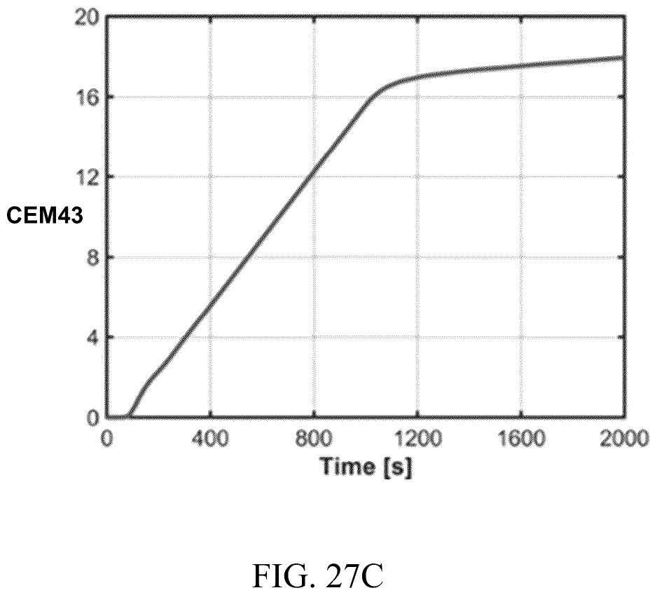

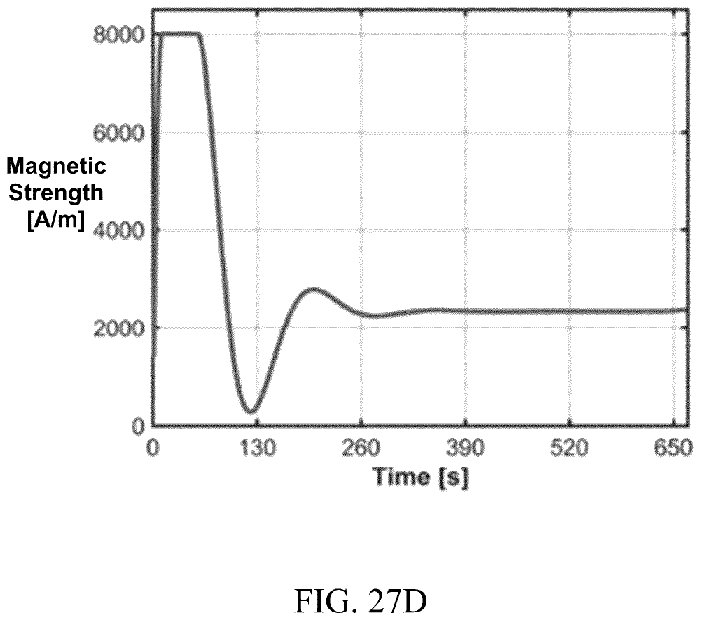

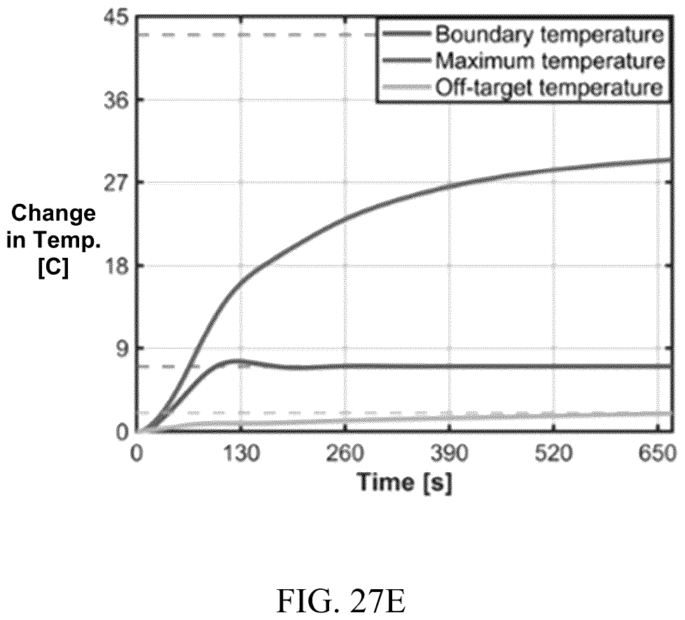

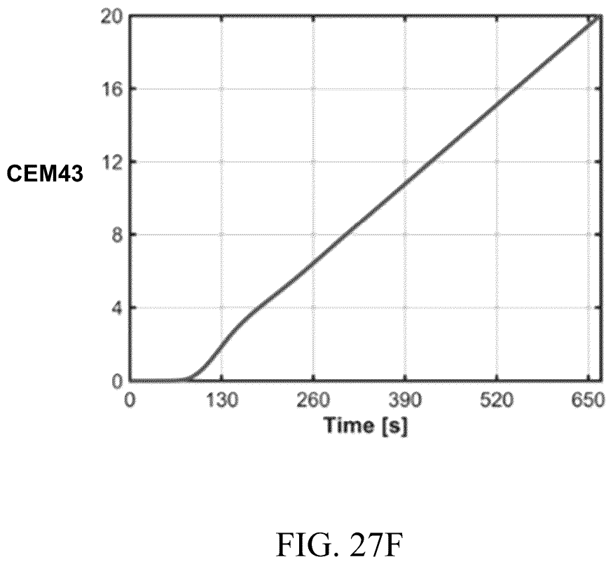

The desired thermal dose was not achieved when the tumor boundary temperature was low; however, at higher tumor boundary temperatures, the desired thermal dose was successfully obtained as shown in FIGS. 27A-F.

It should be understood that modifications to the embodiments disclosed herein can be made to meet a particular set of design criteria. For instance, the number of or configuration of components or parameters may be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as may come within the true scope of this invention, which is to be given the full breadth thereof. Additionally, the disclosure of a range of values is a disclosure of every numerical value within that range, including the end points. Thus, while certain exemplary embodiments of the apparatus and process and/or utilization and methods of making and using the same have been discussed and illustrated herein, it is to be distinctly understood that the invention is not limited thereto but may be otherwise variously embodied and practiced within the scope of the following claims.

What is claimed is:

1. A method for cancer treatment, comprising:
delivering magnetically susceptible materials to a tumor site;
applying a magnetic field to the tumor site to generate heat from the magnetically susceptible materials, thereby heating the tumor site; and
controlling the magnetic field using a controller operatively coupled to a magnetic field generation device that applies the magnetic field, wherein the controller is configured to maintain a first prescribed temperature at a tumor of the tumor site and a second prescribed temperature at healthy tissue adjacent to the tumor site.

2. The method of claim 1, wherein the first prescribed temperature is a temperature greater than or equal to 43° C. and the second prescribed temperature is a temperature that is less than or equal to 39° C.

3. The method of claim 1, wherein the step of applying the magnetic field to the tumor site to generate heat from the magnetically susceptible materials further comprises:
supplying a thermal dose to the tumor, wherein the thermal dose is defined by:
$$CEM 43 = {\int }_{t = 0}^{t = final} {B}^{( \frac{43 ° C . - T ( x , y , z , t )}{1 ° C .} )} dt$$
wherein CEM43 is the thermal dose, t is time, T(x, y, z, t) is a factor related to the temperature dependence of the rate of cell death, T is the temperature, and x, y, z are spatial coordinates.

4. The method of claim 1, wherein the step of controlling the magnetic field using a controller further comprises:
providing the controller with a tumor boundary temperature set point;
measuring a boundary temperature at a tumor boundary of the tumor site;
comparing the tumor boundary temperature set point and the measured boundary temperature to obtain an error; and
adjusting the alternating magnetic field based on the error.

5. The method of claim 4, wherein the tumor boundary temperature set point is 43° C. or 43.5° C.

6. The method of claim 4, wherein the boundary temperature is measured with a temperature probe positioned at the tumor boundary of the tumor site or a scanner device.

7. The method of claim 1, wherein the controller further comprises a fuzzy logic controller.

8. The method of claim 1, wherein the controller is configured to utilize a data driven model to maintain the first prescribed temperature and the second prescribed temperature.

9. A system for cancer treatment, comprising:
a magnetic field generator configured to generate a magnetic field to heat magnetically susceptible materials located adjacent to and/or at a tumor site;
a controller operatively coupled to the magnetic field generator so that the magnetic field is generated to maintain a first prescribed temperature at a tumor of the tumor site and a second prescribed temperature at healthy tissue adjacent the tumor site.

10. The system of claim 9, wherein the first prescribed temperature is a temperature greater than or equal to 43° C. and the second prescribed temperature is a temperature that is less than or equal to 39° C.

11. The system of claim 9, further comprising:
a temperature probe operatively connected to the controller, wherein the temperature probe is configured to measure a boundary temperature at the tumor boundary and transmit the measured boundary temperature to the controller.

12. The system of claim 11, wherein the controller is configured to compare the measured tumor boundary temperature with a tumor boundary temperature set point to obtain an error and adjust how the magnetic field generator generates the magnetic field based on the error.

13. The system of claim 12, wherein the tumor boundary temperature set point is 43° C. or 43.5° C.

14. The system of claim 11, wherein the controller further comprises a fuzzy logic controller.

15. A method for cancer treatment planning, comprising:
delivering magnetically susceptible materials to a tumor site;
imaging the tumor site with a first scanner to acquire tumor data, wherein the first scanner is a computed tomography scanner or a magnetic resonance imaging scanner;
imaging the tumor site with a second scanner to acquire magnetically susceptible materials distribution data and heating rate data, wherein the second scanner is a magnetic particle imaging scanner;
analyzing the tumor data, magnetically susceptible materials distribution data, and heating rate data to simulate a temperature distribution in the tumor site to adjust application of a magnetic field to the magnetically susceptible materials within a body having the tumor site to maintain a first prescribed temperature at the tumor site and a second prescribed temperature at healthy tissue adjacent the tumor site.

16. The method of claim 15, wherein the analyzing of the tumor data, magnetically susceptible materials distribution data, and heating rate data includes using finite element analysis, and the method further comprises:
segmenting the tumor data to obtain segmented tumor data;
segmenting the magnetically susceptible materials distribution data to obtain segmented distribution data; and
co-registering the segmented tumor data and the segmented distribution data.

17. The method of claim 15, wherein the first prescribed temperature is a temperature greater than or equal to 43° C. and the second prescribed temperature is a temperature less than or equal to 39° C.

18. The method of claim 17, also comprising:
providing the controller with a tumor boundary temperature set point;
simulating a boundary temperature at a tumor boundary of the tumor site; and
comparing the tumor boundary temperature set point and the simulated boundary temperature to obtain an error for use in adjusting the application of the magnetic field to the magnetically susceptible materials within the body having the tumor site.

19. The method of claim 18, wherein the tumor boundary temperature set point is 43° C. or 43.5° C.

20. The method of claim 15, wherein the controller further comprises a fuzzy logic controller.
